(19) United States
(12) Patent Application Publication
(10) Pub. No.: US 2021/0335486 A1
(43) Pub. Date: Oct. 28, 2021

(54) SYSTEM AND METHOD FOR GRAPHICAL USER INTERFACE MANAGEMENT PROVIDING HISTORICAL DATA-BASED FORECASTING FOR CLINICAL TRIALS

(71) Applicant: Greenphire, Inc., King of Prussia, PA (US)

(72) Inventors: Kyle Russell Cunningham, Schwenksville, PA (US); Ryan Patrick Kelly, Bryn Mawr, PA (US); Todd Michael Horning, Phoenixville, PA (US); Catherine Alyssa Click, Apex, NC (US)

(21) Appl. No.: 17/243,064

(22) Filed: Apr. 28, 2021

Related U.S. Application Data
(60) Provisional application No. 63/016,704, filed on Apr. 28, 2020.

(51) Int. Cl. G16H 40/20; G06F 16/23; G06Q 10/04; G16H 10/20; G06Q 30/02; G06Q 10/10; G06Q 20/08; G06Q 30/04; G16H 70/20

(52) U.S. Cl. CPC G16H 40/20; G06F 16/2379; G06Q 10/04; G16H 10/20; G06Q 30/0206; G06F 3/0482; G06Q 20/085; G06Q 30/04; G06Q 10/109; G16H 70/20; G06Q 10/103

(57) ABSTRACT

A system and method for controlling a computerized device's display to provide a compact and informative graphical user interface providing historical data-based forecasting for clinical trial-related activities. The graphical user interface performs guided prompting for development of clinical trial plans on a per-study basis, historical data-based budgeting on a per-country basis, and budget negotiation on a per-supplier basis, to effectively form clinical trial agreements. Further, the system provides a high degree of flexibility and feedback to the user during these activities, including via graphical user interfaces providing mechanisms for accessing historical payment data and/or fair market value data derived therefrom, to allow for comparison and/or selection of countries/suppliers to be used in clinical trials and/or to negotiate clinical trial budgets based on actual historical payment data. The system thereby acts as a knowledge base for storing and displaying data relating to a single sponsor's and competing sponsors' past activities.

### CROSS-REFERENCE TO RELATED APPLICATION

This application claims the benefit of priority, under the 35 U.S.C. 119(e) to U.S. Provisional Patent Application No. 63/016,704, filed Apr. 28, 2020, the entire disclosure of which is hereby incorporated herein by reference.

### FIELD OF THE INVENTION

The present invention relates generally to clinical trials, and more particularly to a computer-implemented system and methods for graphical user interface management providing historical data-based forecasting for planning and administration of clinical trial-related activities.

### DISCUSSION OF RELATED ART

Companies in the life sciences industry, such as pharmaceutical, biotechnology, and medical device companies, are generally required to perform clinical trial studies. The purpose of these studies includes testing the efficacy and safety of new life sciences products on human subjects. Many clinical trial studies are conducted in whole or in part outside the United States. In the United States, clinical trial studies and associated data must be reviewed by the Food and Drug Administration (FDA). Clinical trial data may be submitted to a foreign counterpart of the FDA to gain foreign approval for a new life sciences product.

Clinical trial studies tend to be complex logistically, and a single clinical study may involve the activities of many individuals and entities around the world. For example, each clinical trial study typically involves a life science company's (referred to as the “sponsor” of the trial) use of many different suppliers to operate clinical trial “sites” at which individual patients (referred to as “participants”) will receive treatment or otherwise be involved in the clinical trial activities, under the control and/or supervision of a “principal investigator” (PI) and/or other healthcare professionals.

Another aspect of the complexity of such studies is related to tracking of activities of the various parties involved, and of related expenses, and the management of payments among parties involved in the studies. Generally, these studies are operated according to a clinical trial agreement (CTA), which defines tasks//work/milestones, etc., and corresponding payments that the sponsor will make for each of those activities. The tasks/work/milestones to be performed in accordance with the study are sometimes referred to as “deliverables”, which are essentially the goods and services that need to be provided to perform a clinical trial study. The deliverables include goods and services provided at patient sites, as well as sites remote from the patient sites, such as lab and diagnostic sites, or sites where investigators perform services.

Generally speaking, suppliers generate invoices for their services and submit them to the sponsor, or the sponsor's agent, for payment. The sponsor is generally responsible for tracking, managing, and paying appropriate invoices from the approved suppliers for work done in a clinical trial. Greenphire, Inc. of King of Prussia, Pa. provides a commercially-available eClinicalGPS® computerized clinical trial payment management system that provides enhanced tracking of clinical trial activities to facilitate proper and accurate payments to suppliers. This system provides near real-time visibility regarding the work and deliverables that have actually been completed, and helps to ensure accurate and efficient invoicing and payment in accordance with applicable CTAs, to approved suppliers. Generally, clinical trial payment management systems seek to ensure that payments are made for each deliverable in accordance with the CTA-prescribed payment for completion of each deliverable.

Generally, particularly with US-based life sciences companies, the CTAs define tasks (and associated payments) at a site level. This means for example, that a CTA may provide for payment to a particular principal investigator (PI) or hospital as the clinical trial site, when the hospital completes a certain task defined by the CTA, such as a doctor's visit and blood test. For completion of that task, the CTA may provide for a fixed payment, such as $150. Accordingly, as part of making payments and preparing to make payments in accordance with CTAs, clinical trial payment management systems accumulate significant amounts of data as to actual negotiated payment values for payments to various suppliers for various clinical trial related tasks, which may be in various countries, for various CTA tasks.

However, there are limitations to such systems. These systems are generally limited in capabilities in that they essentially provide a mechanism for making a defined payment to a particular party subject to completion of an associated task, obtaining of prescribed approvals, etc. for an existing clinical trial. They provide essentially no functionality relating to planning and/or developing a strategy for performance of a future clinical trial.

What is needed is a clinical trial forecasting system for planning and administration of clinical trial-related activities.

### SUMMARY

The present invention provides a clinical trial forecasting system for planning and administration of clinical trial-related activities. More particularly, the present invention provides a computer-implemented system and methods for graphical user interface management providing historical data-based forecasting for planning and administration of clinical trial-related activities, e.g., to compare and/or select countries and/or suppliers to be used in the performance of a clinical trial and/or to establish and/or inform negotiations of budgets for performance of clinical trials based on actual historical data relating to costs of clinical trial activities associated with various countries and/or suppliers.

### BRIEF DESCRIPTION OF THE FIGURES

The foregoing and other aspects of the present invention will be understood from the following detailed description when read in connection with the accompanying drawings. For the purpose of illustrating the invention, there are shown in the drawings embodiments that are presently preferred, it being understood, however, that the invention is not limited to the specific instrumentalities disclosed. Included in the drawings are the following Figures in which:

FIG. 1 is a system diagram showing an exemplary network computing environment in which the present invention may be employed;

FIG. 2 is a schematic block diagram of an improved Clinical Trial Forecasting system in accordance with an exemplary embodiment of the present invention;

FIG. 3 is a flow diagram illustrating an exemplary method of graphical user interface management providing historical data-based forecasting for clinical trials; and

FIGS. 4A-4AZ illustrate exemplary graphical user interfaces (GUIs) for clinical trial forecasting in the nature of historical payment data-based planning and negotiation for clinical trial-related activities, according to an exemplary embodiment.

### DETAILED DESCRIPTION

The present invention provides a clinical trial forecasting system and/or an improved clinical trial payment management system providing a workflow-specific graphical user interface that leverages historical clinical trial payment data to provide data-based forecasting for planning and administration, particularly clinical trial strategy and associated budgeting, of clinical trial-related activities. Further, the improved system provides a graphical user interface allowing for development of (master) clinical trial study templates defining a visit schedule, procedures etc. based on the study's protocol, development of country templates including country-specific budget items for the elements in the clinical trial study template, and then development of supplier-specific negotiation templates based on the country-specific budget templates. Information is received and displayed in compact, information graphical user interfaces, and budgets and/or negotiations may be conducted in view of compilations of actual and/or estimated fair market values for various tasks, based on actual payment data representing actual prior payments for the same or similar tasks, which can be retrieved and used automatedly without the introduction of human errors in data entry, etc. Approved negotiated values may be communicated to payment systems, and be used for the making of future payments automatedly, without the introduction of human errors in data entry, etc. Templates may be reused and modified as necessary for development of future clinical trial studies, thereby providing knowledge management functionality to the system.

### Network Environment Overview

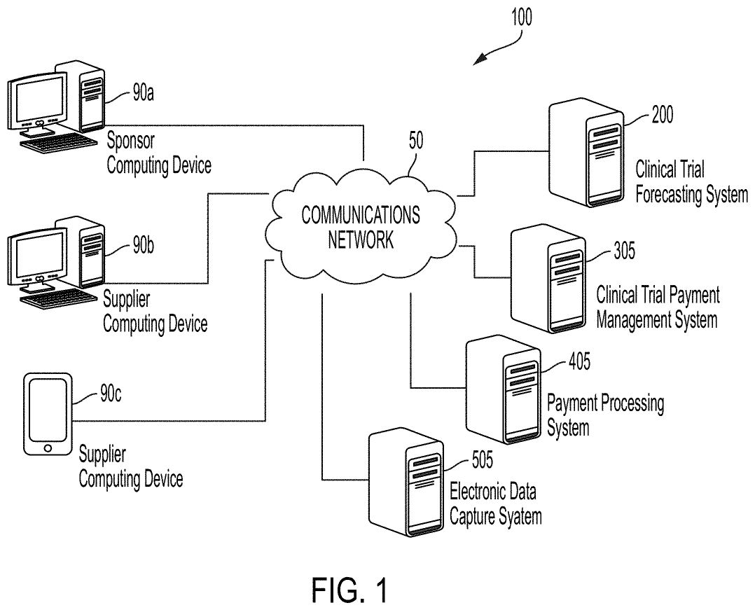

An exemplary embodiment of the present invention is discussed below for illustrative purposes. The present invention may be understood with reference to the exemplary simplified network environment 100 of FIG. 1. As shown in FIG. 1, the exemplary network environment 100 includes computing devices used by suppliers of clinical trial goods and/or services, such as doctor's office, hospital, research center or other clinical trial site personnel. By way of illustrative example, such computing devices may be a desktop computing device 90b or a mobile computing device 90c, such as a smartphone, a tablet computer. Any suitable computing devices may be used for the purposes described herein. By way of example, the desktop computing device 90b may be a personal computer (PC) or the like that includes conventional hardware and software and is able to communicate with a Clinical Trial Payment Management System (CTPMS) 305 for communicating with the sponsor, e.g., via a Sponsor Computing Device 90a and/or suppliers via their computing devices 90b, 90c, for the purpose of managing (including providing instructions to external banking systems, such as Payment Processing System 400 discussed below, for making) payments, as generally known in the art. Each computing device may include conventional hardware and software for communicating via the communications network 50, as generally known in the art. By way of example, the desktop computing device 90b may be disposed within a hospital 20, e.g., at a nurse's station, and the mobile computing device 90c may be transportable for use anywhere at which network connectivity is available. As known in the art, the supplier computing devices 90b, 90c are used to enter data into records of a clinical trial management system and/or Clinical Trial Payment Management System (CTPMS) 305 to record data relevant to conducting and managing the clinical trial, and ultimately for receiving payment for those tasks as defined by an applicable CTA. For example, these devices 90b, 90c may be used to record data reflecting that a particular patient was seen by a particular doctor at a particular hospital on a particular date, and received certain medical care or examinations during that doctor's visit. The CTPMS 305, such as Greenphire's eClinicalGPS® system, may communicate with or be integrated with an otherwise conventional clinical trial management system (CTMS), such as IBM Clinical Development, Edge CTMS, or an electronic data capture (EDC) provider, such as Medidata RAVE, or Oracle InForm, that may be used by clinical trial sites to record/log/report the occurrence of clinical trial activities at a clinical trial site.

Further, the exemplary network environment 100 of FIG. 1, further includes a Payment Processing System (PPS) 400. The PPS 405 is responsible for effectuating the making of payments/monetary transfers, based on payment instructions developed from use of the CTPMS 305, and may be entirely conventional. For example, the PPS 405 may be or include hardware/software/systems responsible for making ACH or SWIFT payments, wire transfers, or other payment transfers. By way of example, banking institutions operate such systems and/or provide commercially-available services involving use of such a system. Any suitable PPS 405 may be used, and hardware, software and systems for implementing the PPS 405 are well-known in the art and beyond the scope of the present invention, and thus are not discussed in detail herein.

In accordance with the present invention, the exemplary network environment 100 further includes a Clinical Trial Forecasting System (CTFS) 200. In some embodiments (not shown), the CTFS 200 structure and functionality may be effectively integrated into the CTPMS 305, but the two are shown separately here for illustrative clarity. The CTFS 200 generally includes conventional hardware and software for communication via the communications network 50, and further includes additional structure and/or functionality in accordance with the present invention, as described herein.

In this exemplary embodiment, the Clinical Trial Forecasting System 200 is operatively connected to at least Sponsor Computing Device 90a and Clinical Trial Payment Management System 300 (which may be in turn operatively connected to the computing devices 90b, 90c and the Payment Processing System 400) via the data communications network 50, such as the Internet and/or via a Virtual Private Network (VPN) connection. Hardware and software for enabling communication of data among the computing devices and system via such communications networks are well known in the art and beyond the scope of the present invention, and thus are not discussed in detail herein.

### Clinical Trial Forecasting System

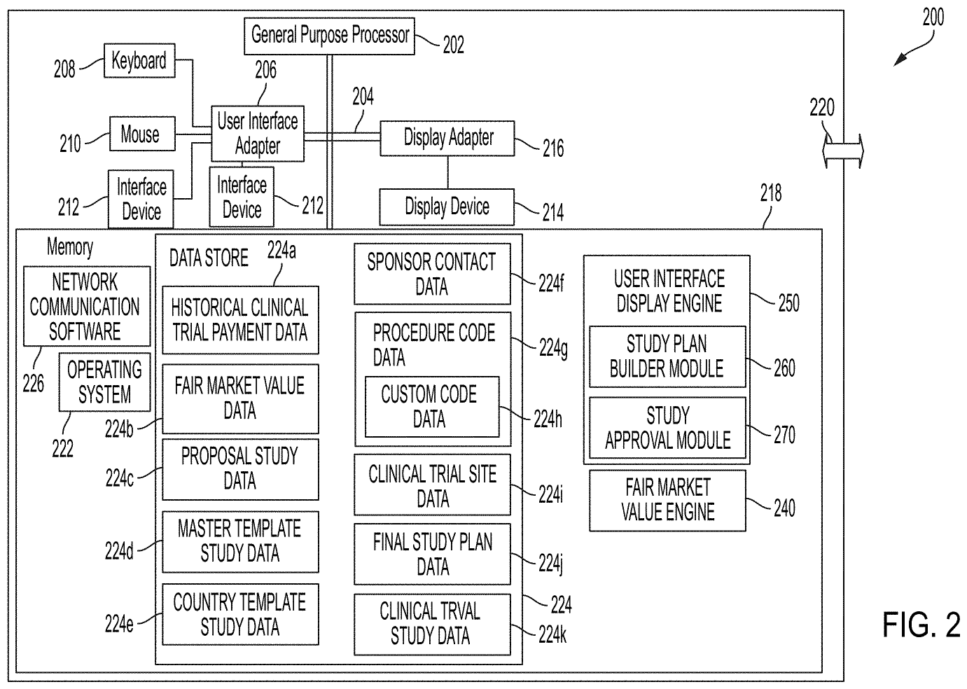

FIG. 2 is a schematic block diagram showing an exemplary Clinical Trial Forecasting System (CTFS) 200 in accordance with an exemplary embodiment of the present invention. This CTFS 200 is a special-purpose computer system that includes conventional computing hardware storing and executing both conventional software enabling operation of a general purpose computing system, such as operating system software 222 and network communications software 226, and specially-configured computer software for configuring the general purpose hardware as a special-purpose computer system for carrying out at least one method in accordance with the present invention. By way of example, the communications software 226 may include conventional web server software, and the operating system software 222 may include iOS, Android, Windows, Linux software.

Accordingly, the exemplary CTFS 200 of FIG. 2 includes a general-purpose processor, such as a microprocessor (CPU), 102 and a bus 204 employed to connect and enable communication between the processor 202 and the components of the CTFS in accordance with known techniques. The exemplary CTFS 200 includes a user interface adapter 206, which connects the processor 202 via the bus 204 to one or more interface devices, such as a keyboard 208, mouse 210, and/or other interface devices 212, which can be any user interface device, such as a touch sensitive screen, digitized entry pad, etc. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The CTFS 200 may communicate with other computers or networks of computers, for example via a communications channel, network card or modem 220. The CTFS 200 may be associated with such other computers in a local area network (LAN) or a wide area network (WAN), and may operate as a server in a client/server arrangement with another computer, etc. Such configurations, as well as the appropriate communications hardware and software, are known in the art.

The CTFS 200 is specially-configured in accordance with the present invention. Accordingly, as shown in FIG. 2, the CTFS 200 includes computer-readable, processor-executable instructions stored in the memory 218 for carrying out the methods described herein. Further, the memory 218 stores certain data, e.g. in one or more databases or other data stores 224 shown logically in FIG. 2 for illustrative purposes, without regard to any particular embodiment in one or more hardware or software components.

Further, as will be noted from FIG. 2, the CTFS 200 includes, in accordance with the present invention, a User Interface Display Engine (UIDE) 250, shown schematically as data stored in the memory 218, which includes a number of additional modules providing functionality in accordance with the present invention, as discussed in greater detail below. These modules may be implemented primarily by specially-configured software including microprocessor—executable instructions stored in the memory 218 of the CTFS 200. The modules of the UIDE 250 are shown logically in FIG. 2 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

As shown in FIG. 2, the CTFS 200 includes not only a User Interface Display Engine (UIDE) 250 in accordance with the present invention, but also stores particular data in the data store in accordance with the functionality provided by the UIDE 250 and its various modules. Optionally, other software may be stored in the memory 218 and and/or other data may be stored in the data store 224 or memory 218.

Referring now to FIG. 2, in this example, the CTFS 200 stores historical clinical trial payment data 224a (including amounts of actual prior payments and actual prior negotiated amounts of payments reflected in actual clinical prior trial agreements, even if those payments were never actually made) in the data store 224. For example, such historical clinical trial payment data may include information identifying actual payments made in association with various clinical trial studies that have already been completed and/or are in progress. Accordingly, such historical clinical trial payment data may identify the identities of various tasks/deliverables to be performed pursuant to various studies, the names of suppliers to whom those payments were made, an identification of countries in which those suppliers exist and/or in which services were provided, and/or the clinical trial phase and indication/purpose/nature of the study, e.g., a Phase III multiple myeloma clinical trial study. For example, the historical clinical trial payment data may indicate that for completion of an initial patient intake visit for a particular sponsor's clinical trial, the principal investigator/supplier was to be paid a payment of $100 in one country, and $75 in another country for the same/similar task. Other tasks/deliverables and associated payments may be identified for the same and other clinical trials in the historical clinical trial payment data. The historical clinical trial payment data may be retrieved/received from the Clinical Trial Payment Management System 300, and be stored in the CTFS 200. Alternatively, the historical clinical trial payment data may not be stored at the CTFS 200, but rather may be stored at the CTPMS 305, and be referenced by the CTFS 200.

The exemplary CTFS 200 further includes a Fair Market Value Engine (FMVE) 240. The FMVE 240 processes original/raw historical payment data received from the Clinical Trial Payment Management System 300 (and optionally stored in the CTFS 200 as historical clinical trial payment data 224a) to create Fair Market Value Data 224b, which is stored in the data store 224 of the CTFS 200. The processing of the historical payment data may be performed according to any desired logic, to develop any suitable aggregation, characterization, or interpretation of the data (collectively referred to herein as “aggregation” for non-limiting purposes only). Each item unit cost may be mapped to a corresponding Clinical Procedural Terminology (CPT) (managed by the American Medical Association/AMA) code or other custom/proprietary code to allow for comparison of unit costs for the same/comparable tasks. Additionally, item unit costs may be tracked by country, by clinical trial phase and/or indication in support of a finer granularity of comparison of units for the same/comparable tasks. For example, raw historical payment data (e.g., raw historical payment data associated with a specific indication of therapy area) may be received from the CTPMS 305, and may be processed to create averages or quartiles, deciles or other statistical percentiles that may be used by the CTFS 200 as fair market value (FMV) data 224b representing the historical clinical trial payment data 224a in a manner more useful for planning/budgeting purposes for future clinical trials. By way of example, the fair market value data 224b may indicate that for completion of an initial patient intake visit for various sponsors' clinical trials, a group of principal investigators/suppliers were paid an average of $125 in one country (or region), and $95 in another country/region for the same/similar task.

Additionally, the FMVE 240 may be configured to process the historical clinical trial payment data 224a to generate relevant estimated FMV data 224b where insufficient historical data exists, to provide fair market value data usable for the purposes described herein. For example, historical clinical trial payment data for a certain task in country A may be processed to provide FMV task data for country B, if no (or statistically insufficient) historical clinical trial payment data for the same tasks exists for country B. By way of example, data from another country deemed to be a similar or comparable market (e.g., Portugal for data needs in Spain) may be used as a substitute, and a conversion for currency differences and/or other factors (e.g., a history of “premium” or “discounted” pricing relative to another country) may be applied.

In this embodiment, the historical clinical trial payment data 224a and the processed fair market value data 224b are stored in the CTFS 200. It will be appreciated that in other embodiments, for example, only the fair market value data 224b (and not the raw historical payment data 224a) may be stored at the CTFS 200, and/or that the CTFS 200 and CTPMS 305 will be integrated into a single system.

In any event, historical clinical trial payment data 224a (from the CTPMS 305) and/or the fair market value data 224b (resulting from processing such raw historical clinical trial payment data) provides historical-based information and reference points for establishing and/or negotiating payment structures and/or budgets for future clinical trials. In other words, the historical clinical trial payment data may provide historical data-based benchmarks representing fair market value for various tasks, based on amounts previously paid for the same or similar tasks, and that fair market value may advantageously be tracked on a per-supplier and/or per-country basis to inform and/or aid in development of a strategy as to where future clinical trial activities may be performed, which suppliers may be used, etc., and to develop a budget for a future clinical trial.

The UIDE 250 is primarily responsible for causing display of graphical user interface windows, e.g., at the Sponsor Computing Device, and for receiving user input via such user interface windows, as described herein. The UIDE 250 includes a Study Plan Builder Module (SPBM) 260. The SPBM 260 performs functions necessary to cause display, at a Sponsor Computing Device 90a, of various graphical user interface windows displaying information, and configured for receiving input, permitting a user (e.g., at a sponsor/pharmaceutical company, etc.) to interface with the CTFS 200 to design/build a plan, or specification, for a clinical trial study. For example, the graphical user interface windows displayed allow the user to interact with the Sponsor Computing Device 90a to define a desired patient visit schedule device, tasks to be performed at each visit or other deliverables that are otherwise as part of the clinical trial, and amount desired to be paid by the sponsor for each such deliverable. Accordingly, this allows the user to specify the most critical clinical trial agreement parameters for a particular clinical trial study.

The UIDE 250 of the CTFS 200 further includes a Study Approval Module (SAM) 270. The SAM 270 performs functions necessary to cause display, e.g. at a Sponsor Computing Device 90a and/or at one or more Supplier Computing Devices 90b, 90c, of various graphical user interface windows displaying information, and configured for receiving input, permitting a sponsor user (e.g., at a sponsor/pharmaceutical company, etc.) and a supplier user (e.g., at a clinical trial supplier, such as a principal investigator and/or staff at a hospital, university, doctor's office, blood test laboratory or other clinical trial site or clinical trial service provider) to interface with the CTFS 200 to review a proposed plan, or specification, for a clinical trial study, and to negotiate and agree upon amounts to be paid by a sponsor to a supplier for performance of tasks to be performed at each patient visit or other deliverables that are otherwise as part of the clinical trial. For example, the graphical user interface windows displayed allows the supplier user to interact with a Supplier Computing Device 90b, 90c to review a desired patient visit schedule, tasks to be performed at each visit or other deliverables that are otherwise as part of the clinical trial, and amounts desired to be paid by the sponsor for each such deliverable, and to provide a counteroffer of corresponding amounts desired to be received by the supplier for each such deliverable, with the goal of the sponsor and supplier continuing negotiations of the clinical trial plan/budget and/or pricing structure until an agreement is reached. Accordingly, this allows the sponsor user and supplier user to negotiate and agree upon the most critical clinical trial agreement parameters for a particular clinical trial study, until the negotiated proposal is approved by the sponsor.

As referred to above, the UIDE 250 provides the graphical user interface allowing a user of a Sponsor Computing Device 90a to define clinical trial deliverables and otherwise build a clinical trial study plan, and for users of a Sponsor Computing Device 90a and a Supplier Computing Device 90b, 90c to negotiate payments for the clinical trial study plan, based on historical clinical trial payment data and/or related fair market value data. Further, the CTFS 200 may be integrated with the CTPMS 305 to obtain/use such historical clinical trial payment data, and/or to make payments to suppliers for a future clinical trial study after a sponsor and supplier have negotiated the clinical trial study plan and sponsor approval has been obtained. It should be noted that the hardware, software and functionality of the CTFS 200 may be combined with that of the CTPMS 305 and/or the Payment Processing System (PPS) 400.

It should be noted that data communication between these systems and/or integration of the CTPMS and CTFS systems provides several advantages. First, the historical clinical trial payment data (and/or related FMV data) will be accurate, as any human error in finding relevant data or inputting the data into a separate system will be avoided, as the data will be tracked accurately with the payment system. Second, FMV data may be created based on a broad, and more complete, set of historical clinical trial payment data, and the FMV data may be used for negotiation purposes, as a result of the systematic capture and processing of such data as compared to any sponsor's ad hoc process. Third, a single sponsor may use historical clinical trial payment data (and/or related FMV data) based not only on that single sponsors activities, but rather based on many different sponsor's activities, as tracked by the CTPMS system. Fourth, the entire negotiation and approval workflow occurs within the system, such that human error in data entry and/or analysis is avoided. Fifth, negotiated payments approved within the CTFS can be automatically communicated to and/or tracked and used by the CTPMS (e.g., within a single system infrastructure) without the introduction of human data entry errors or other errors associated with manual data entry processes between separate data systems. Sixth, actual negotiation payments/rates are automatically provided to the FMV engine to continuously refine/optimize payments/rates in a conceptual feedback loop.

### Graphical User Interface Management

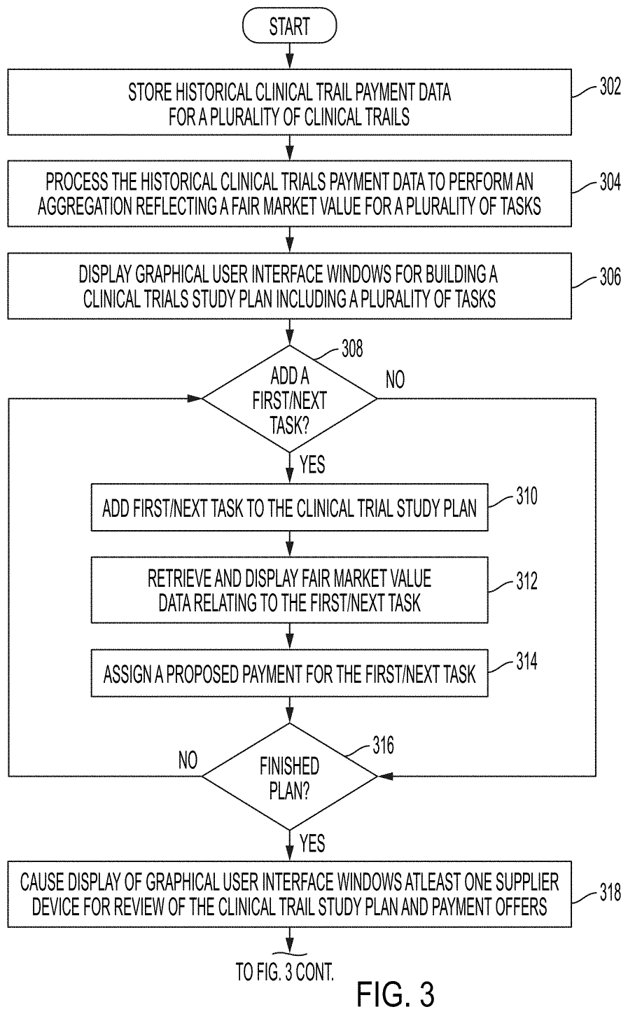

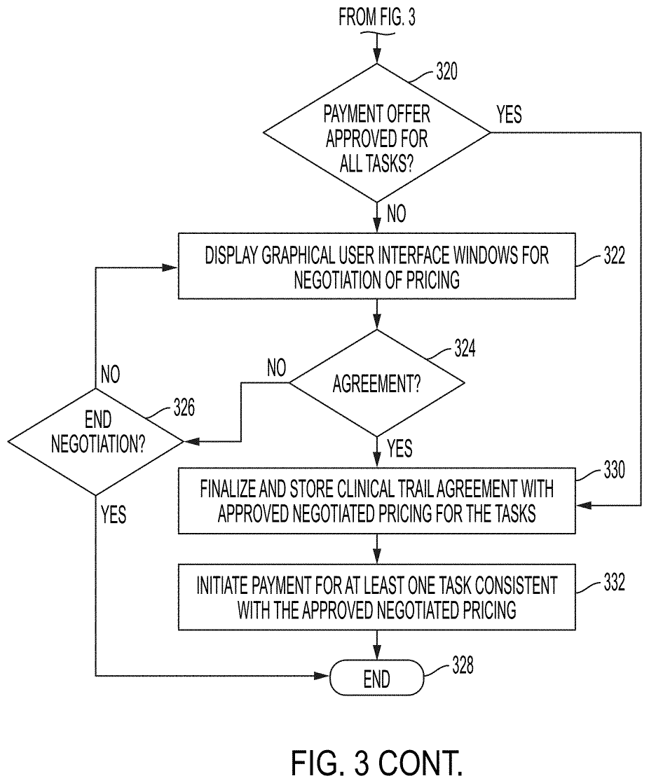

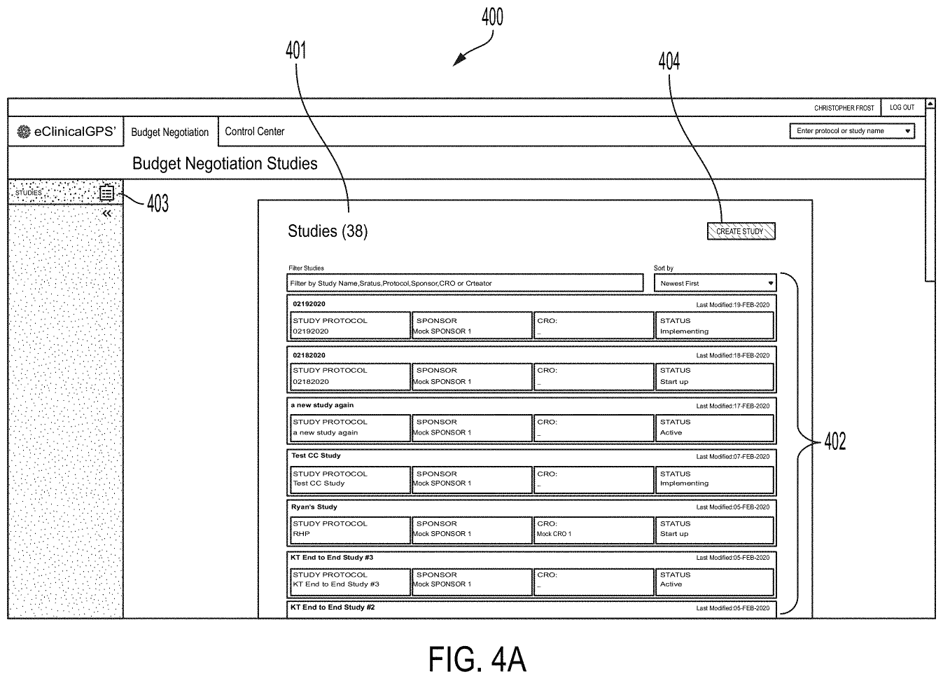

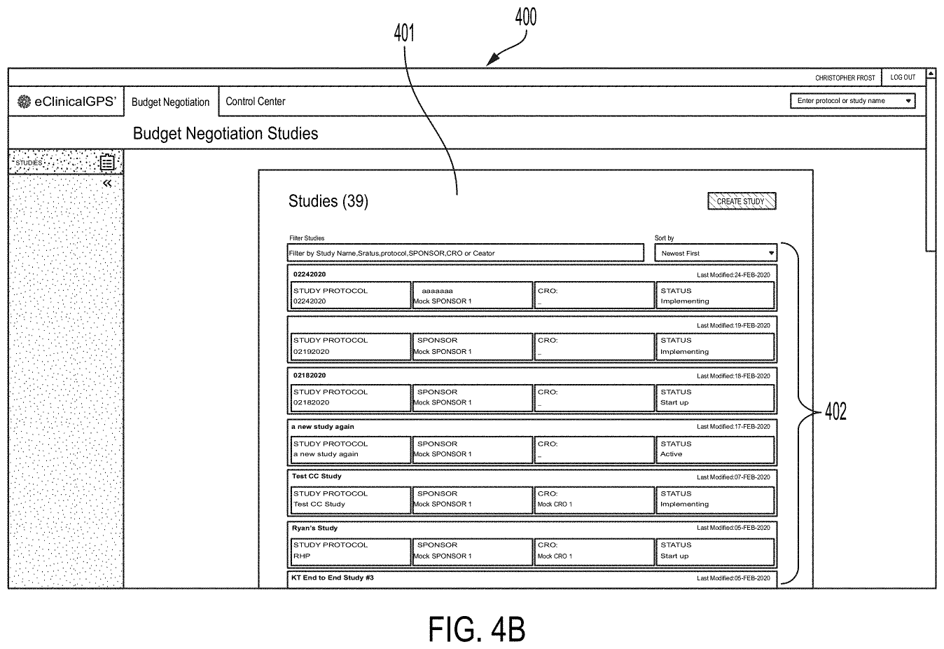

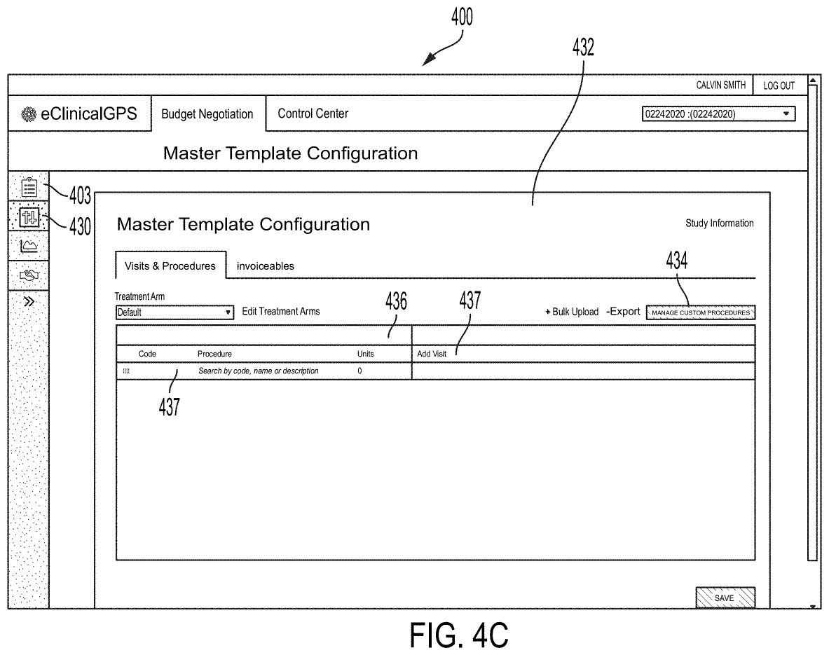

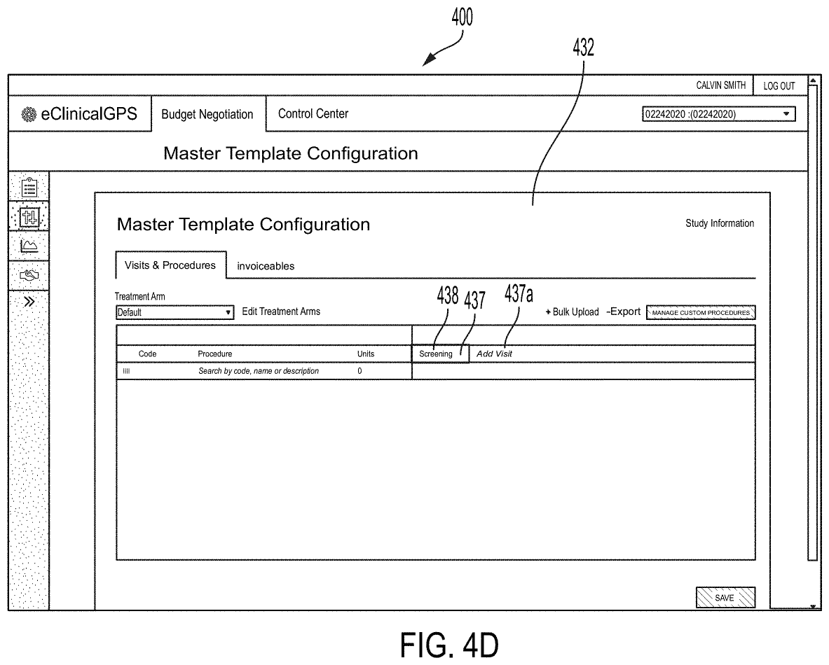

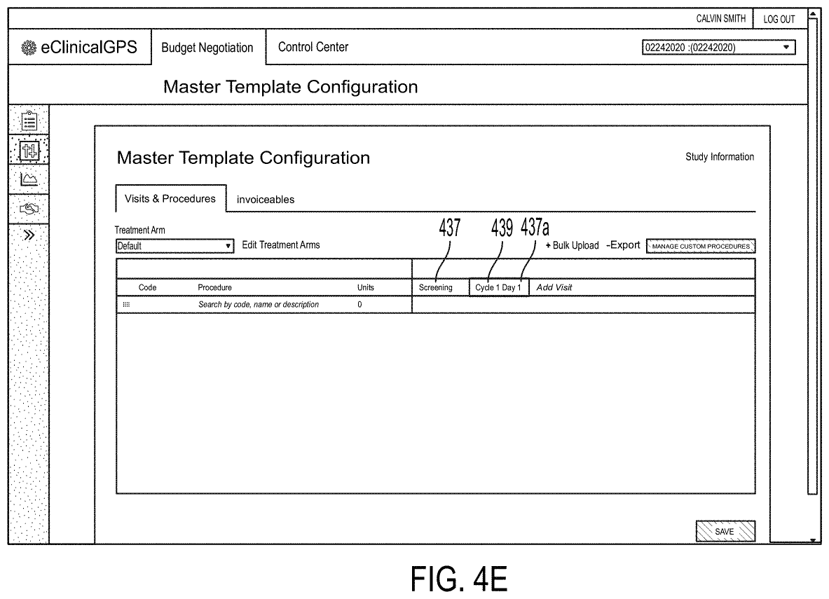

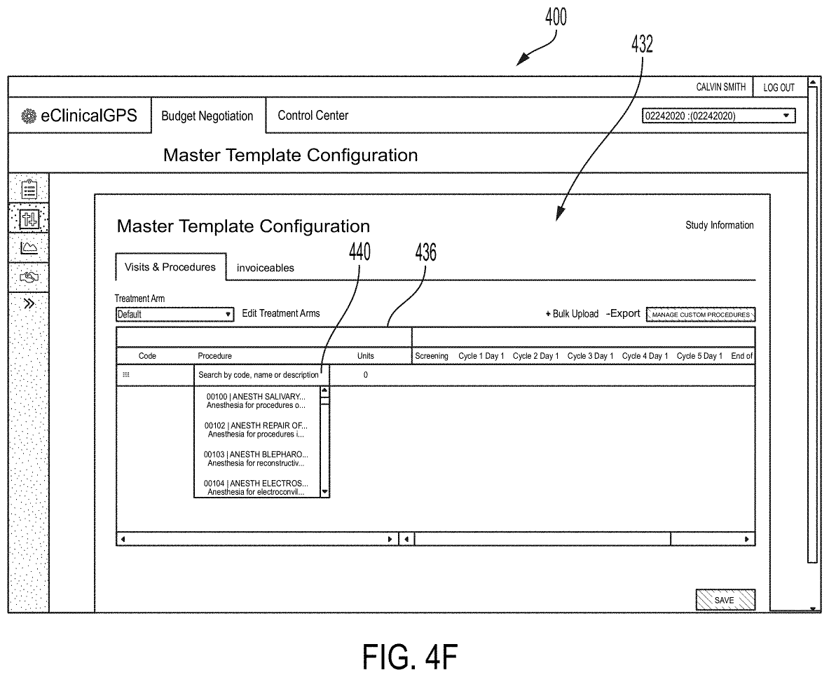

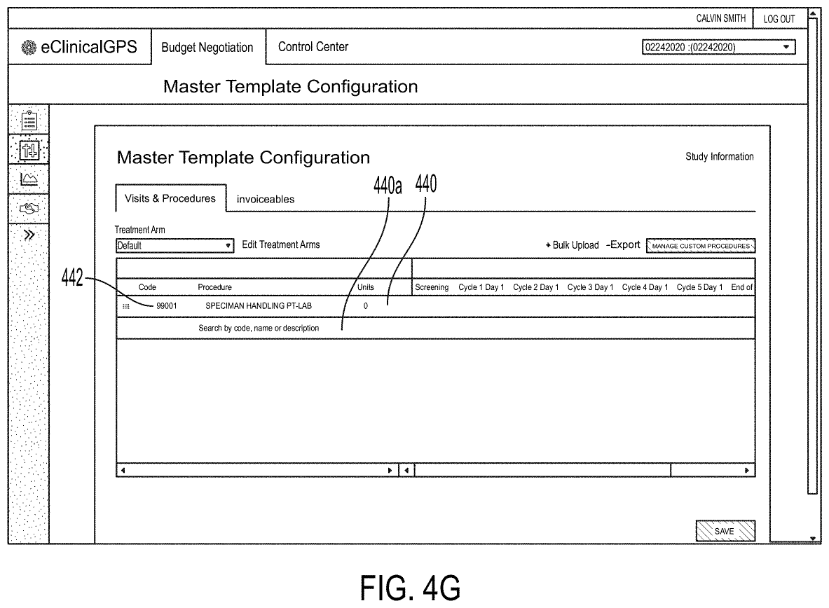

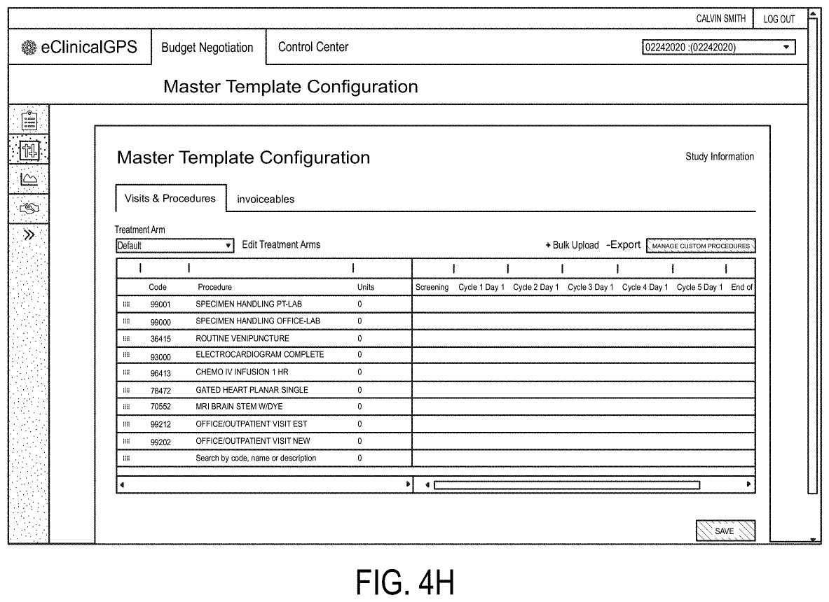

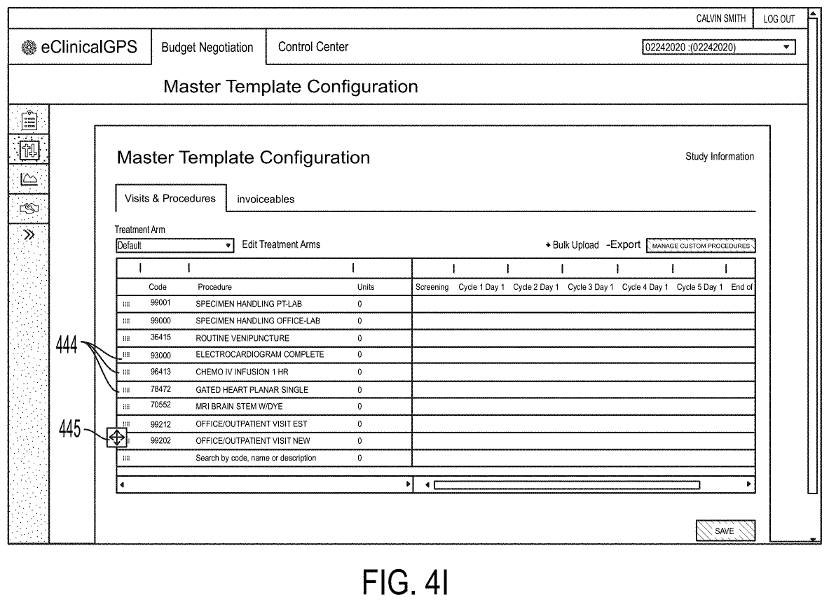

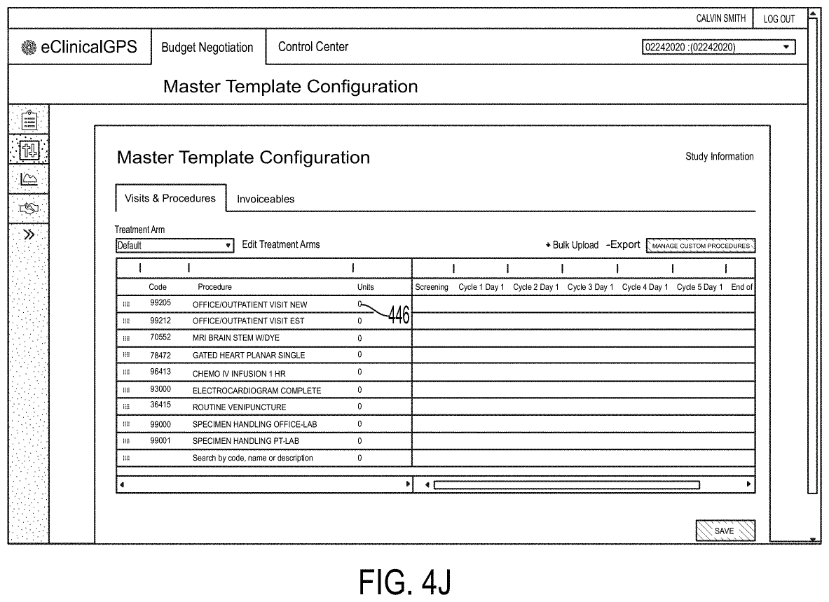

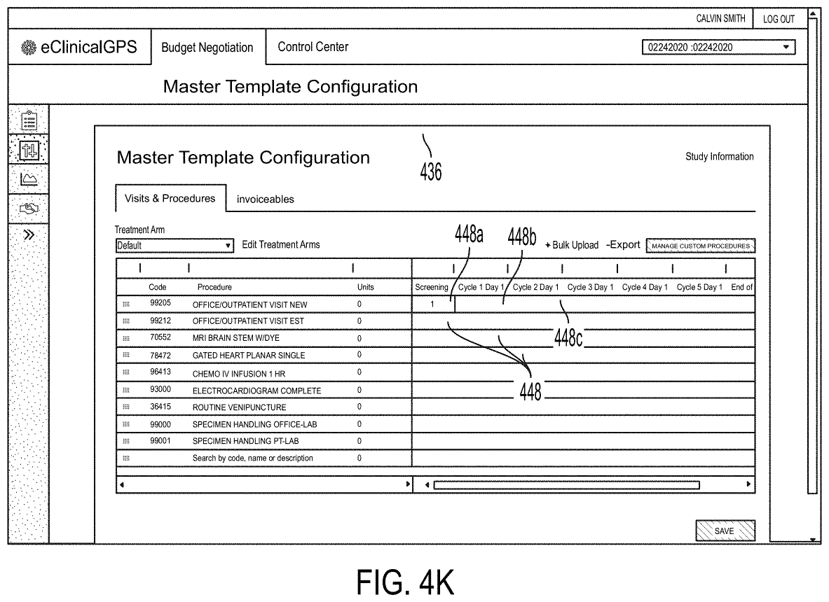

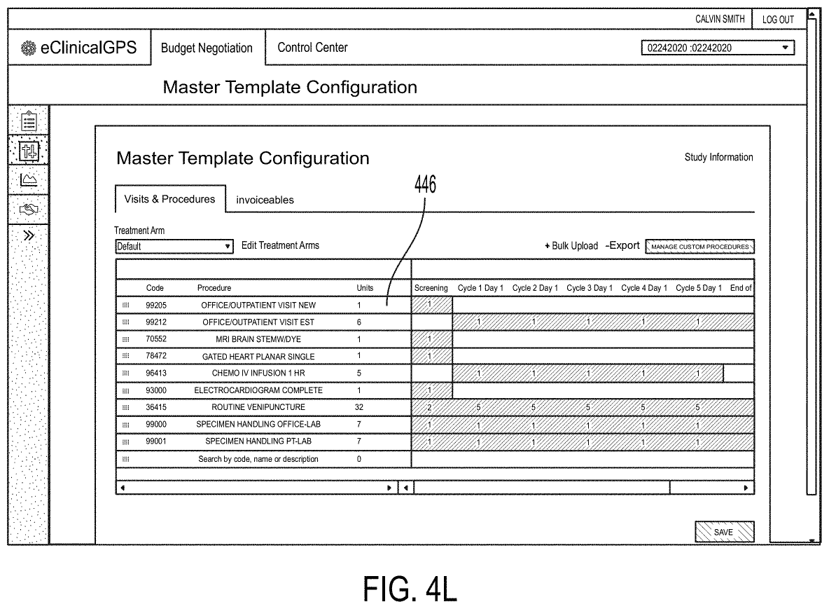

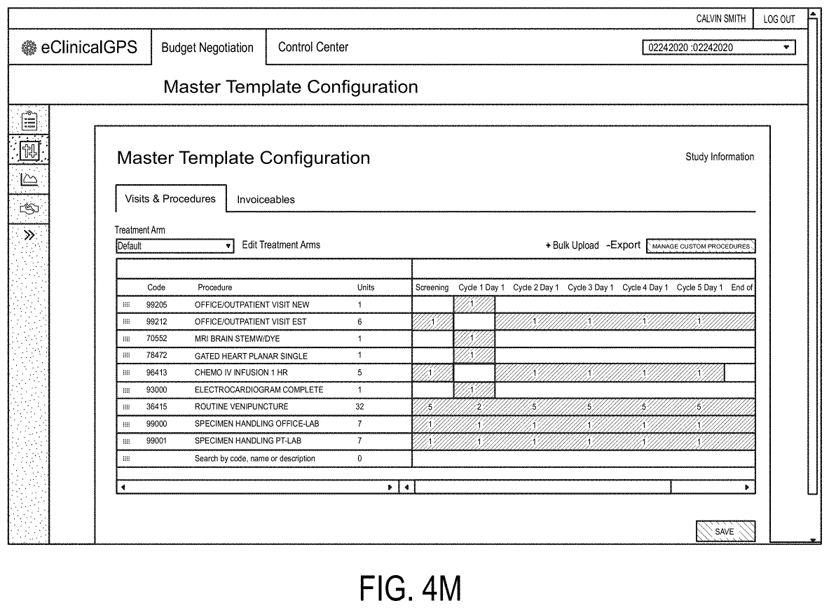

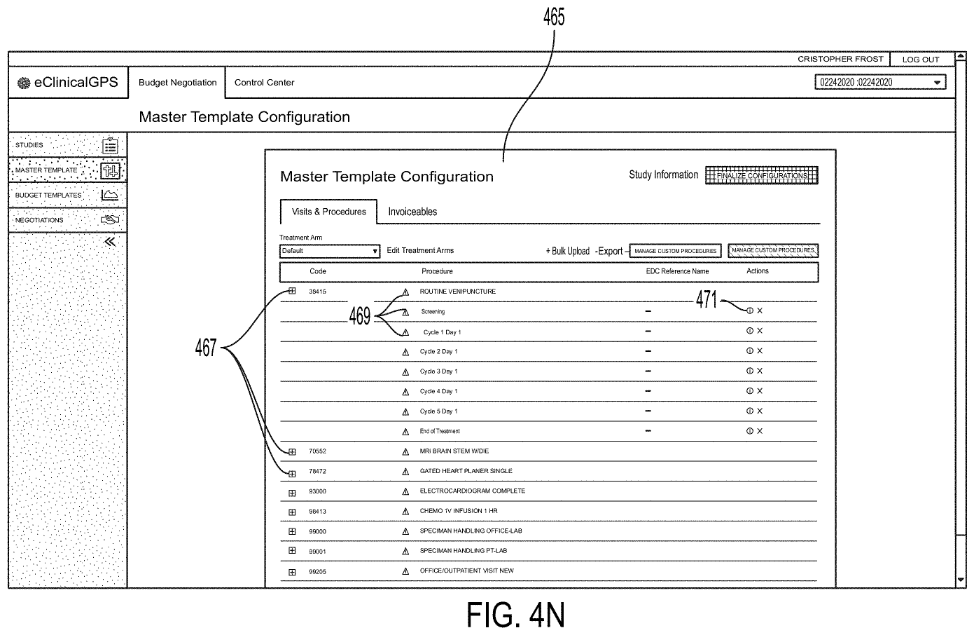

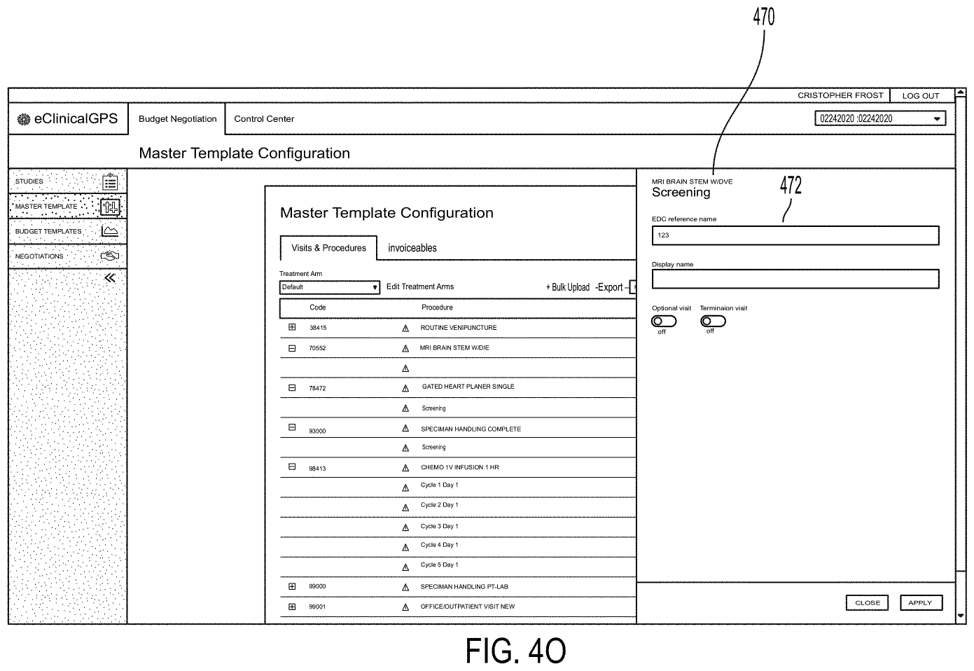

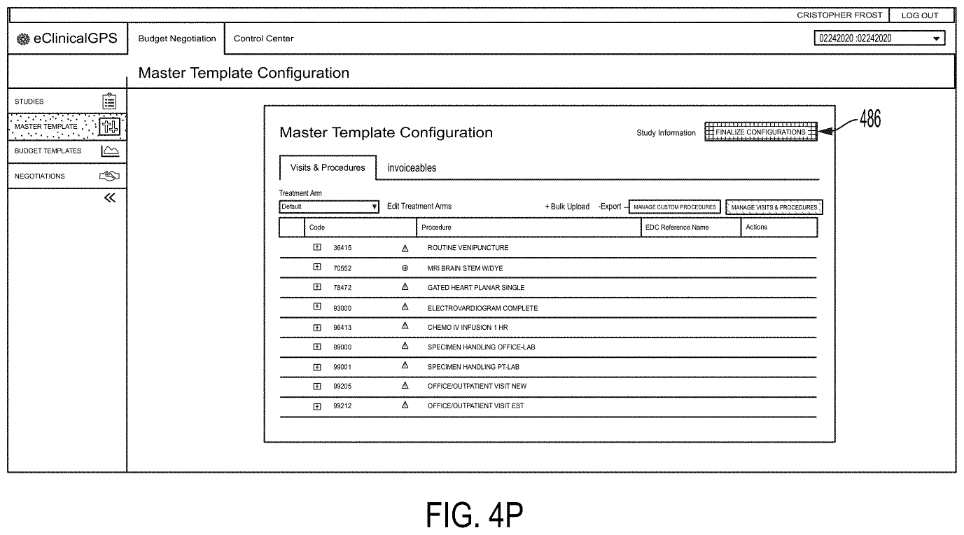

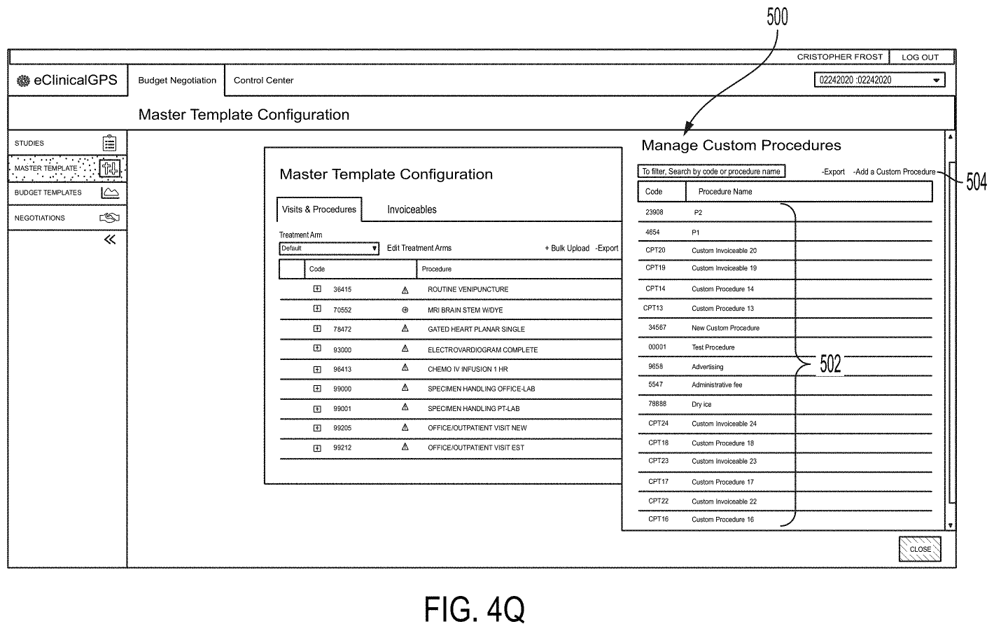

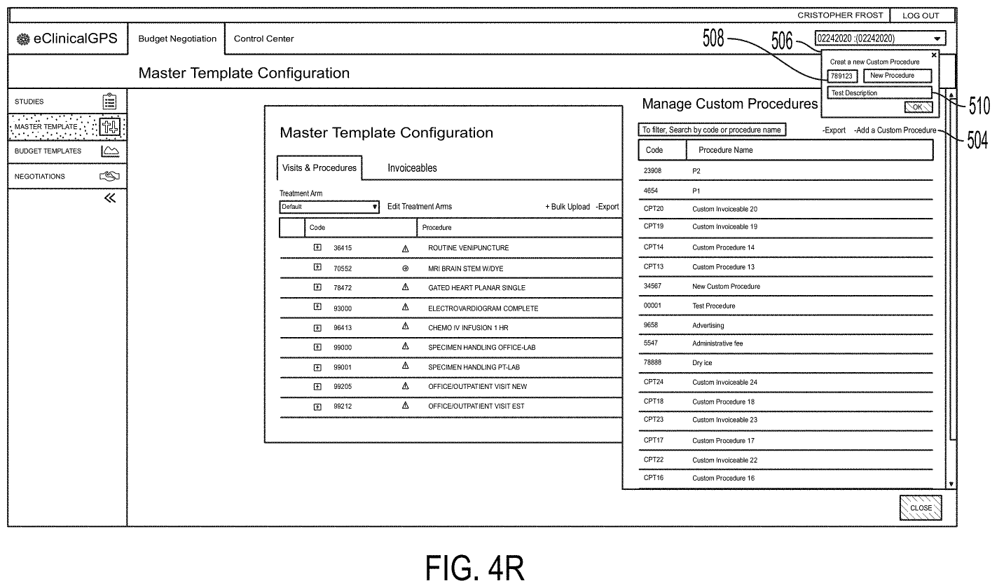

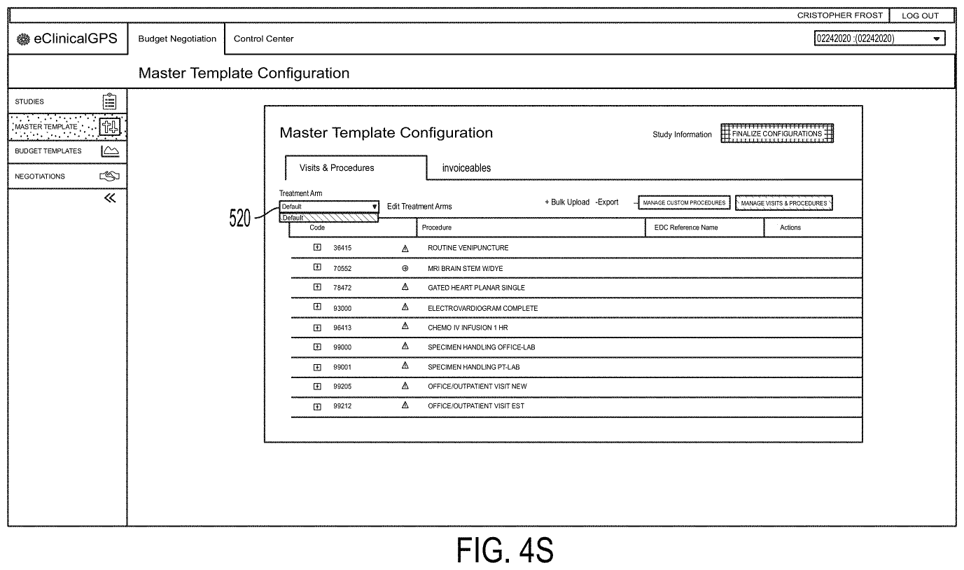

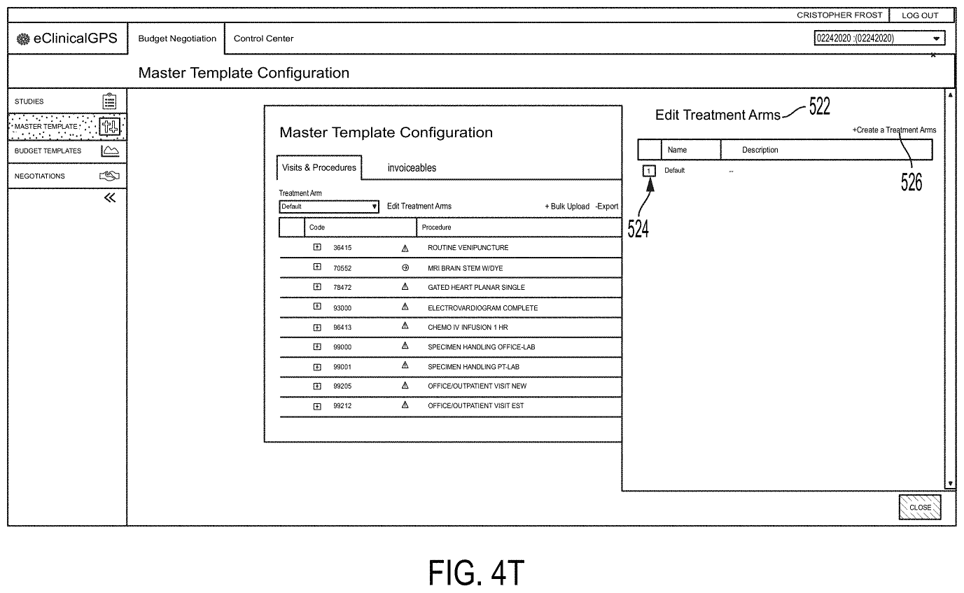

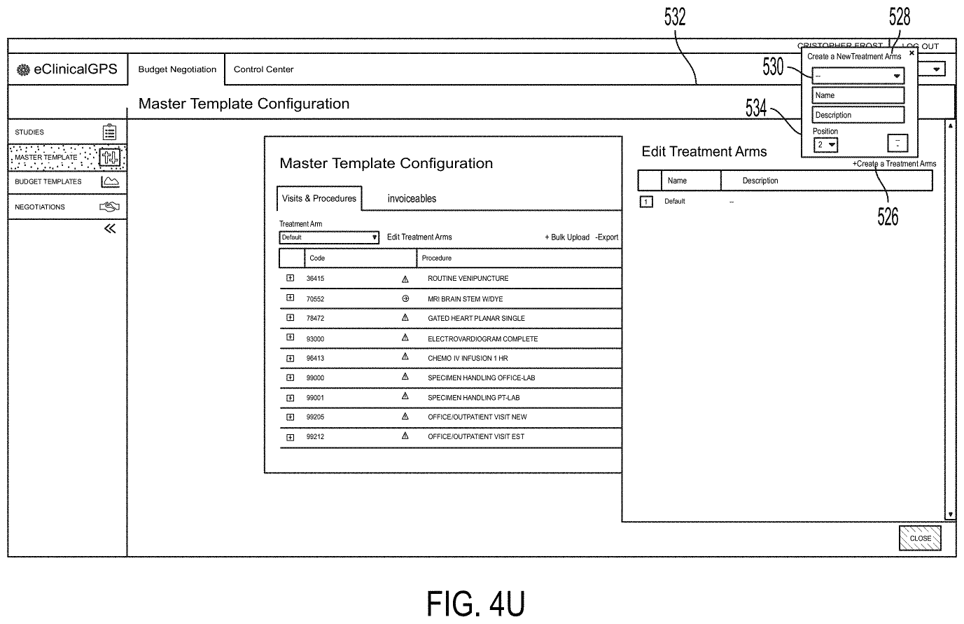

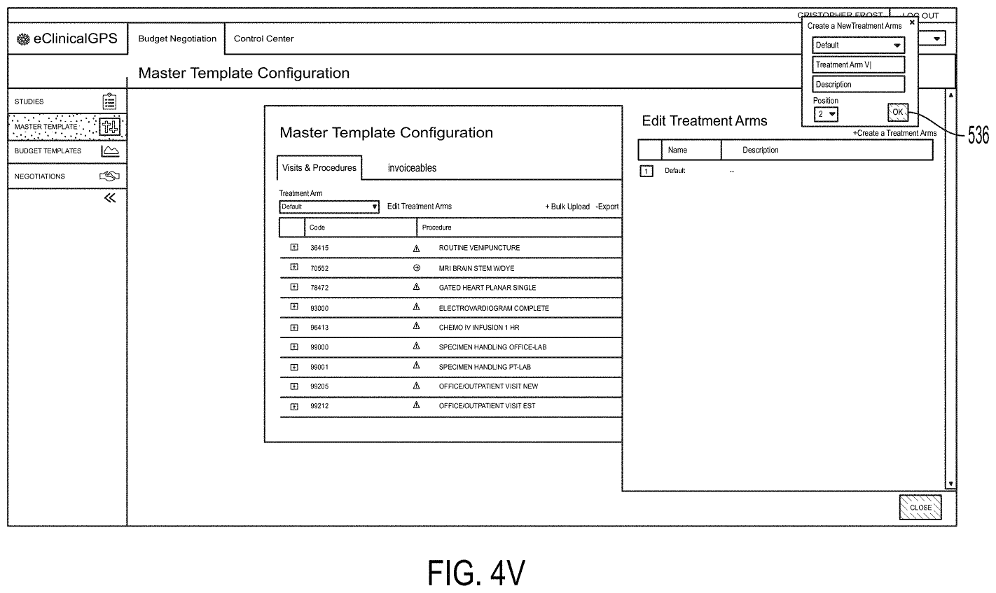

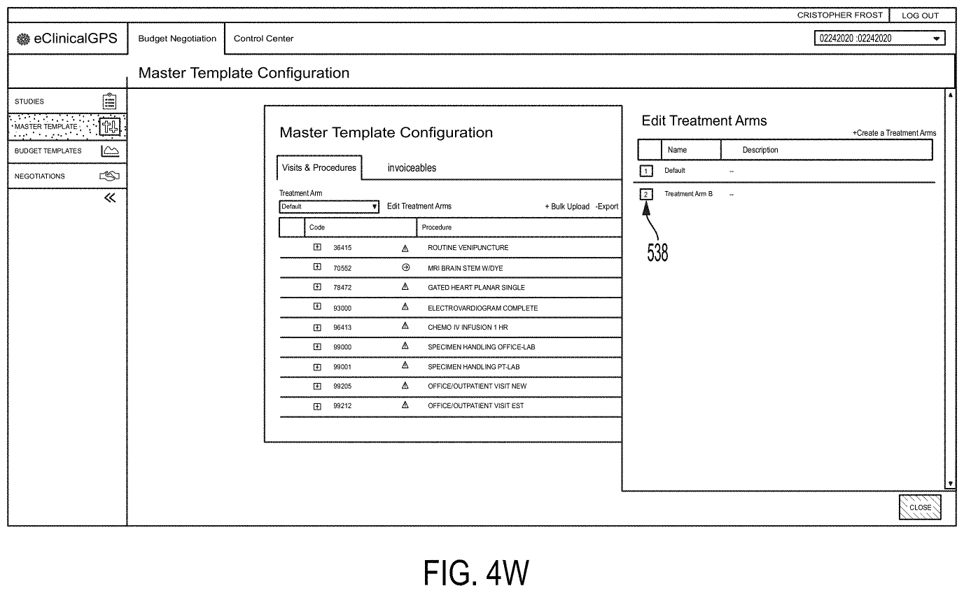

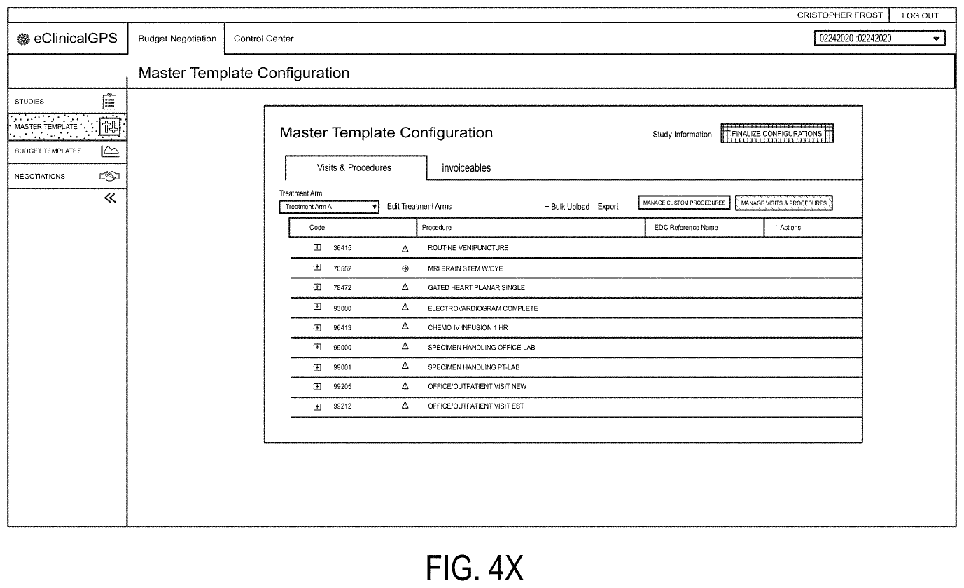

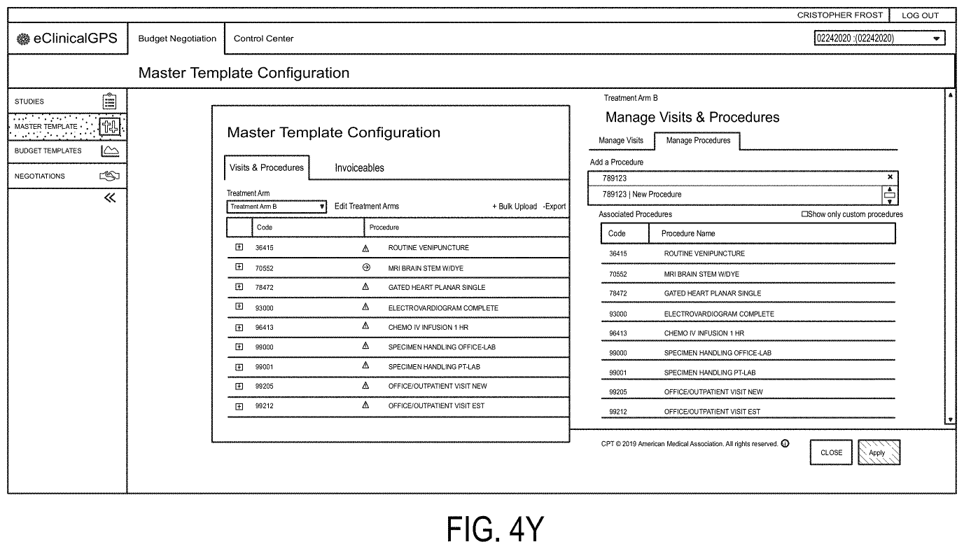

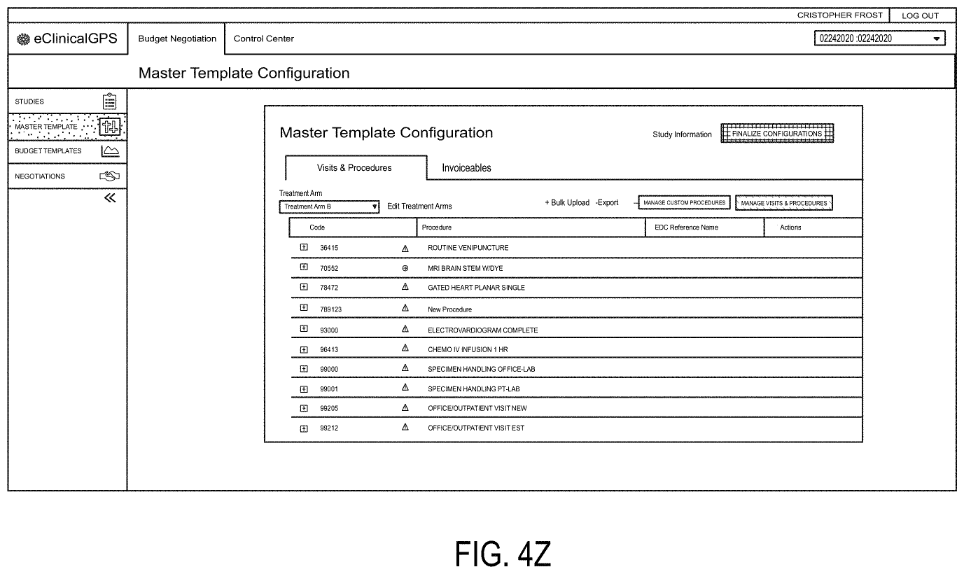

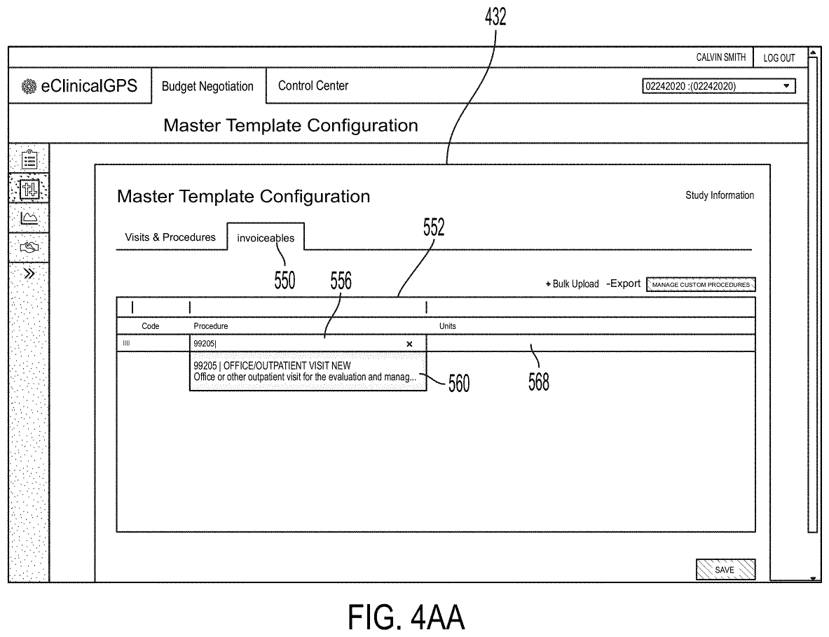

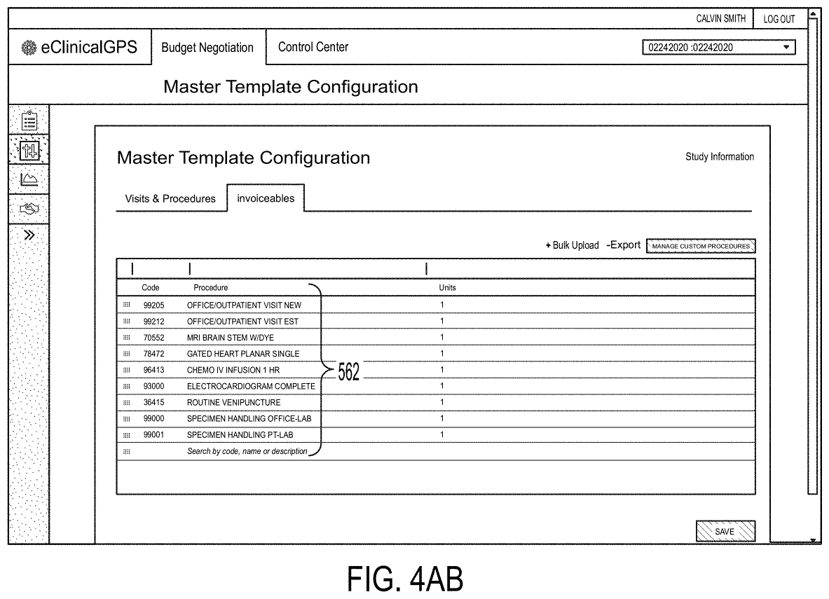

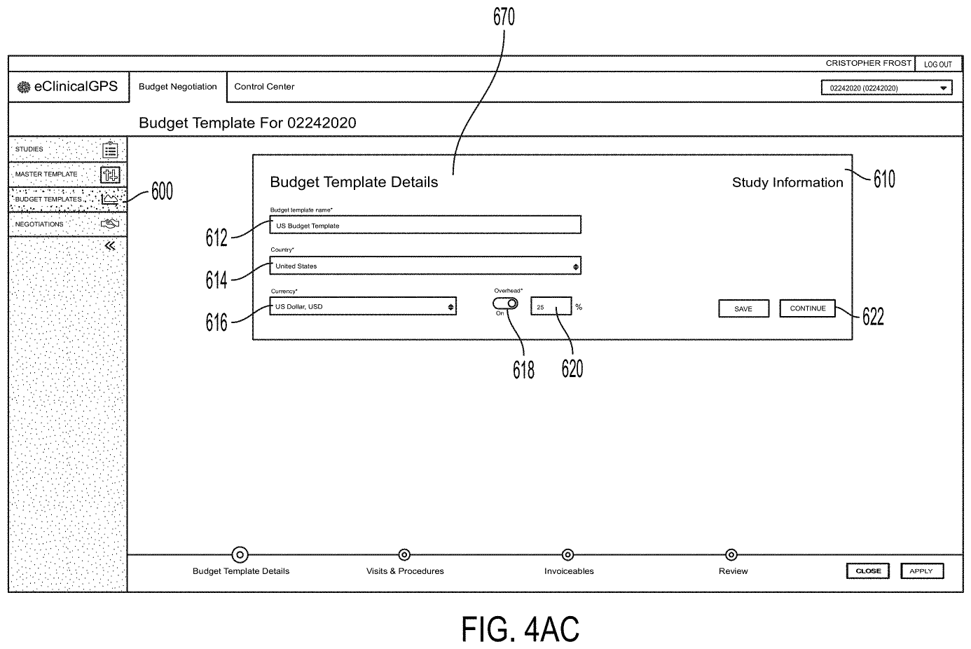

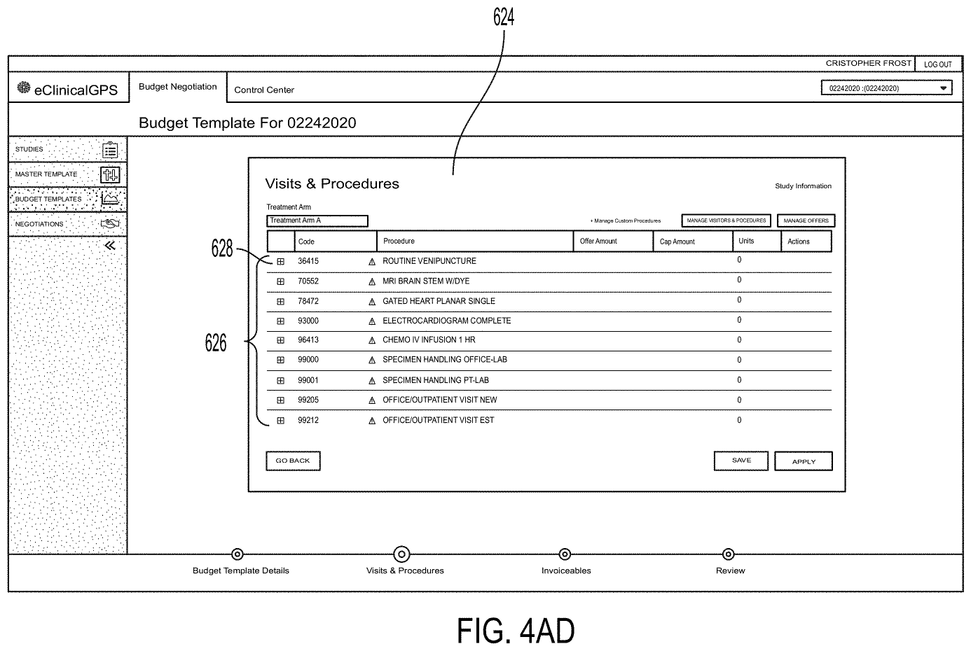

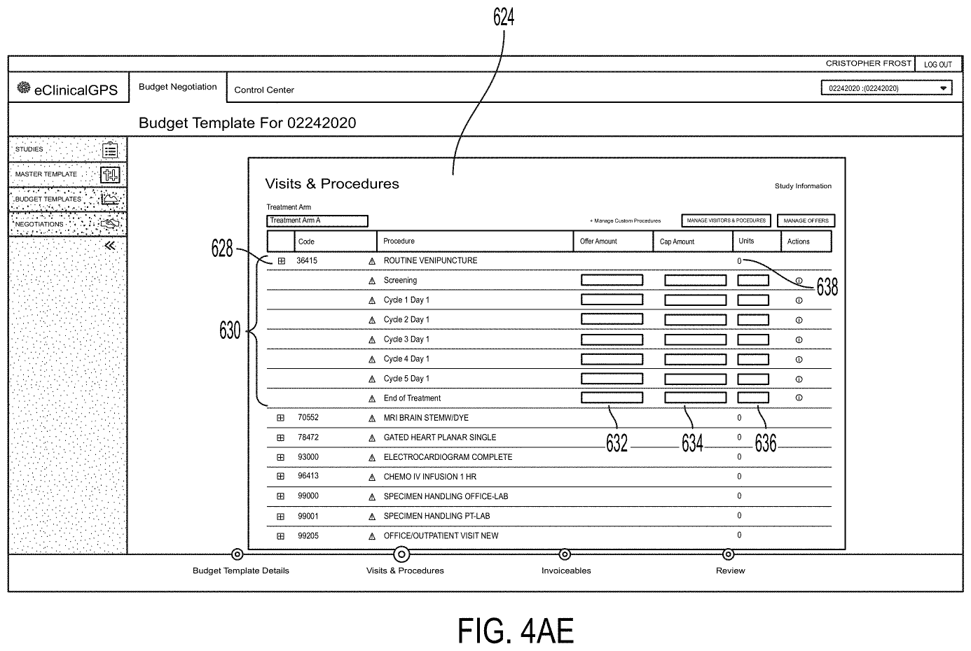

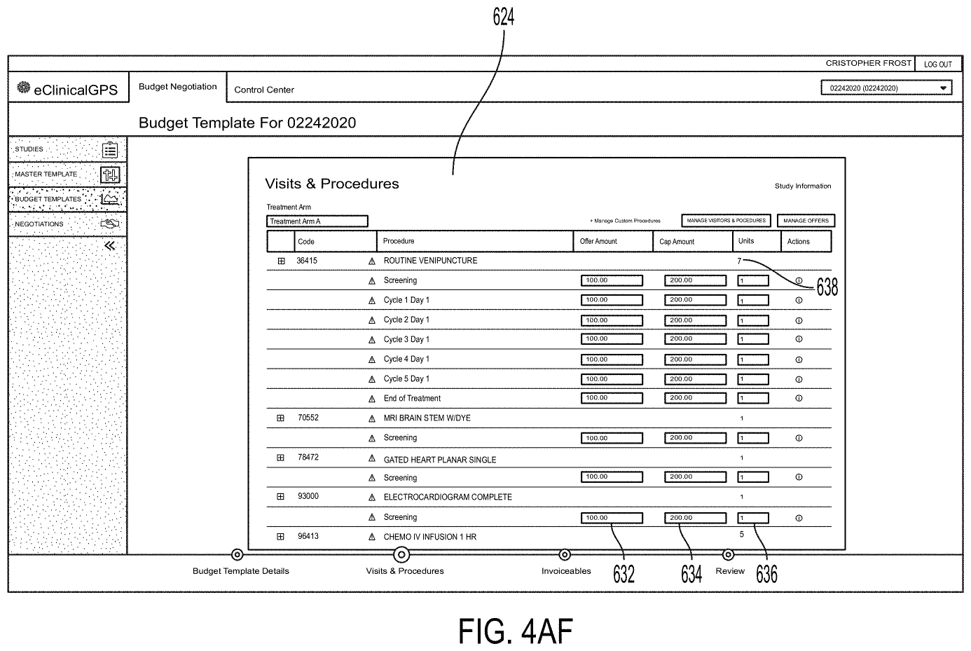

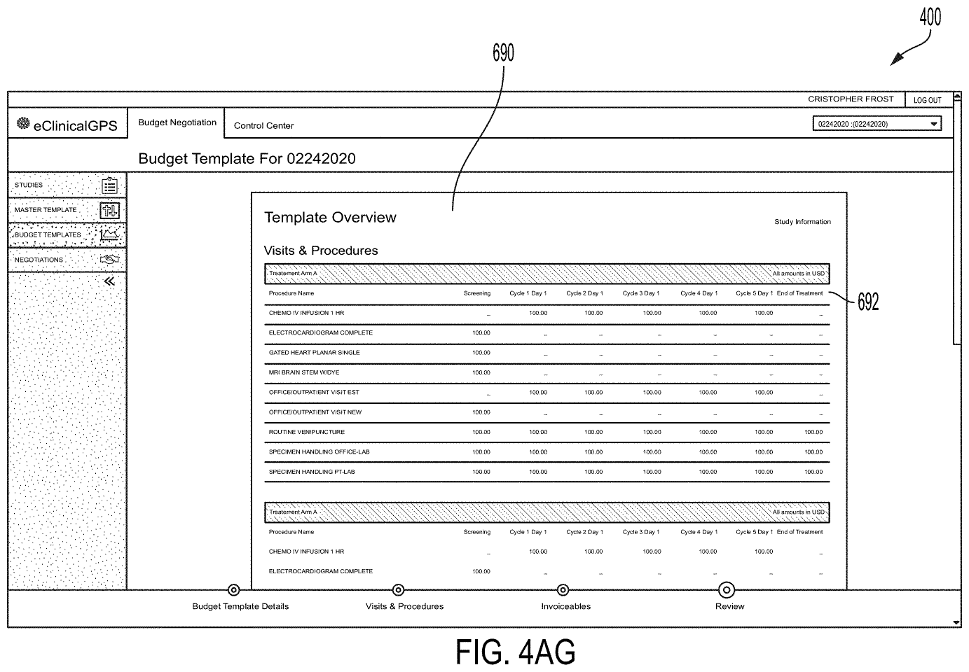

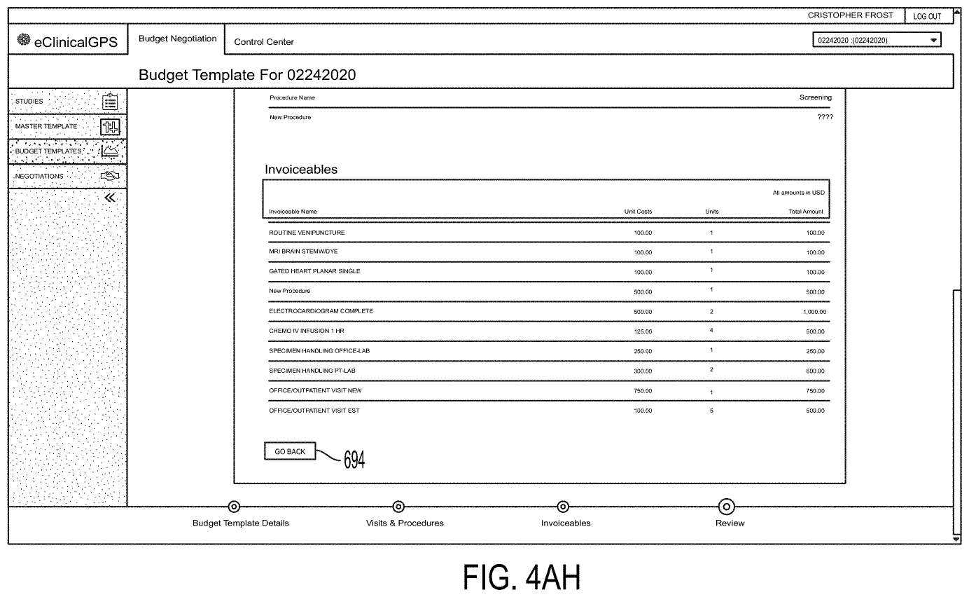

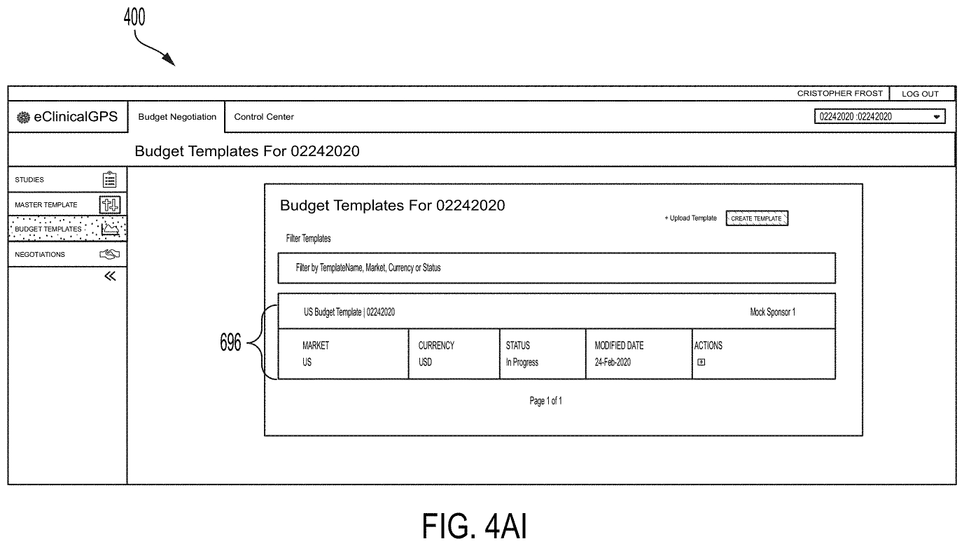

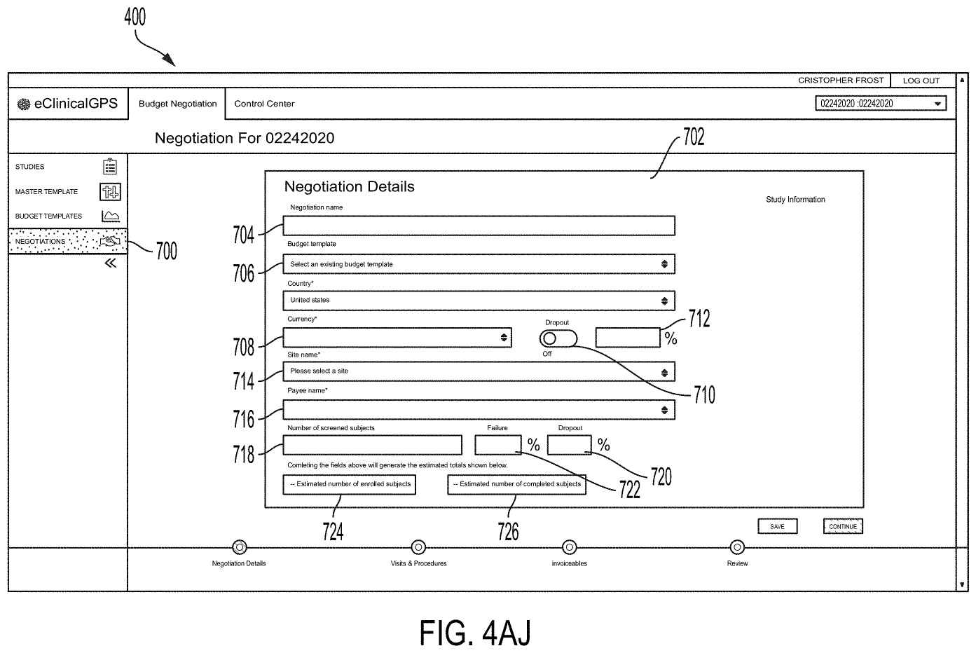

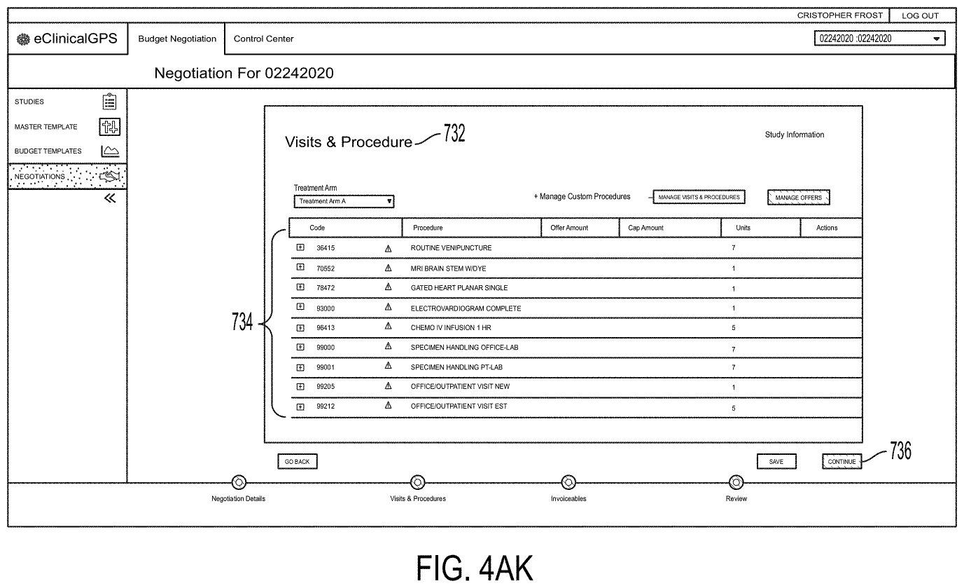

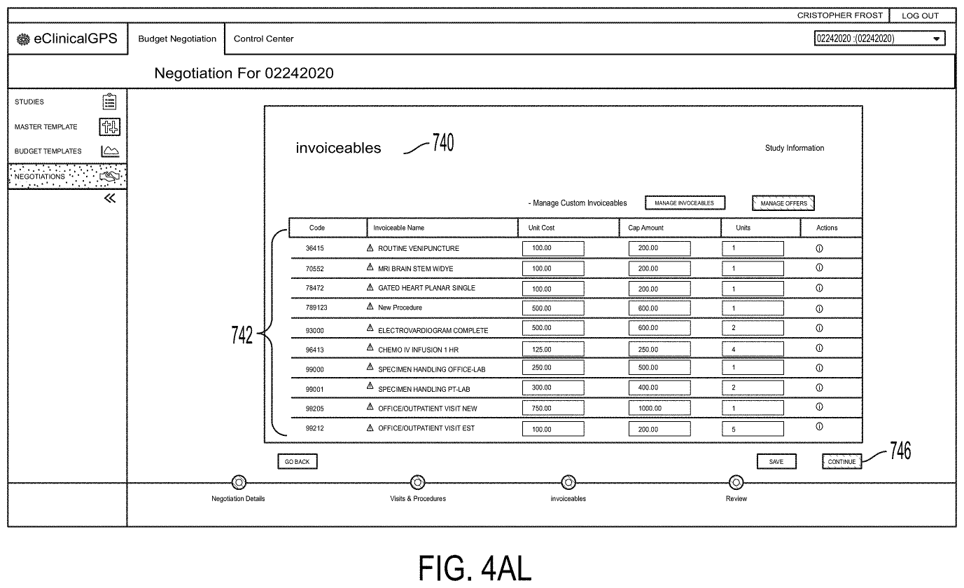

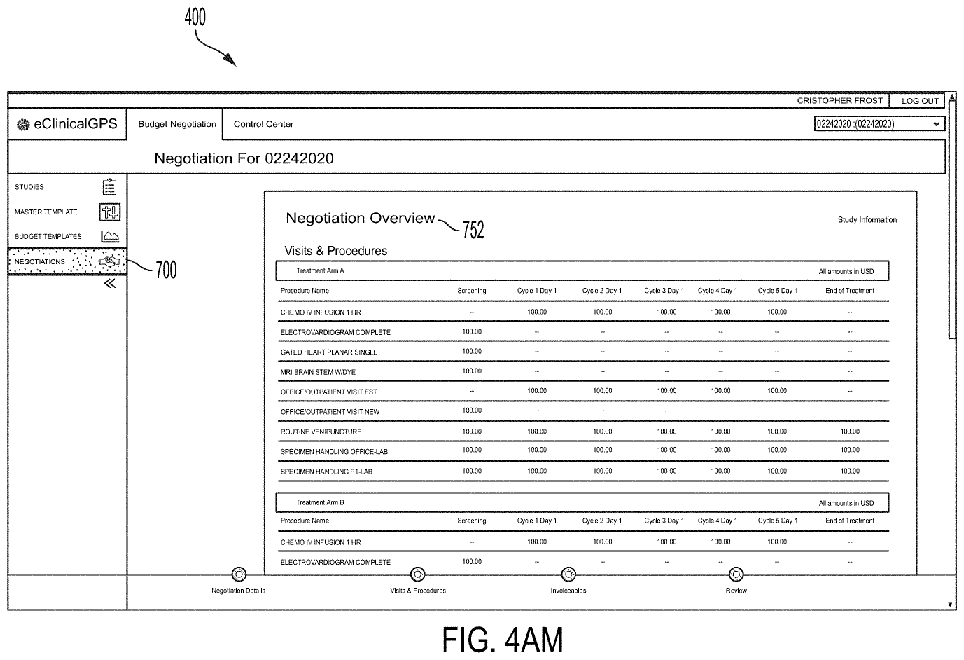

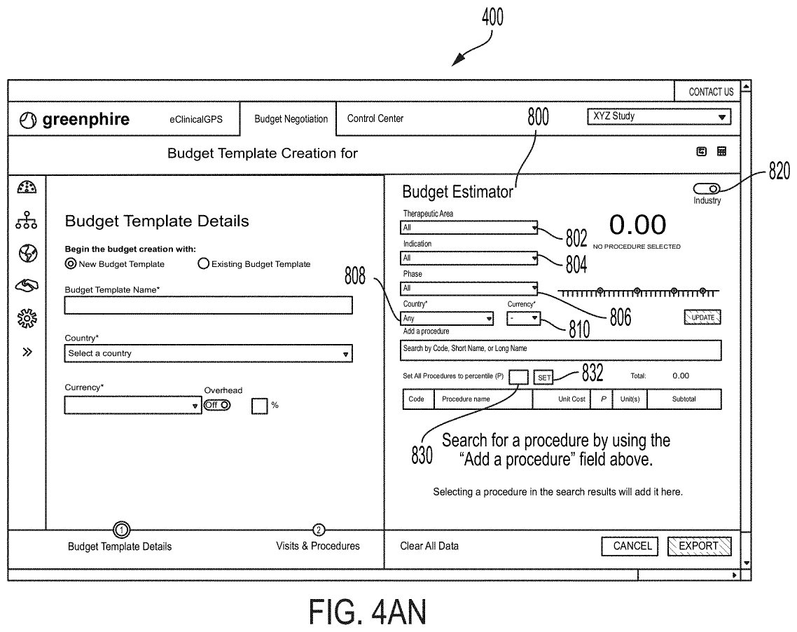

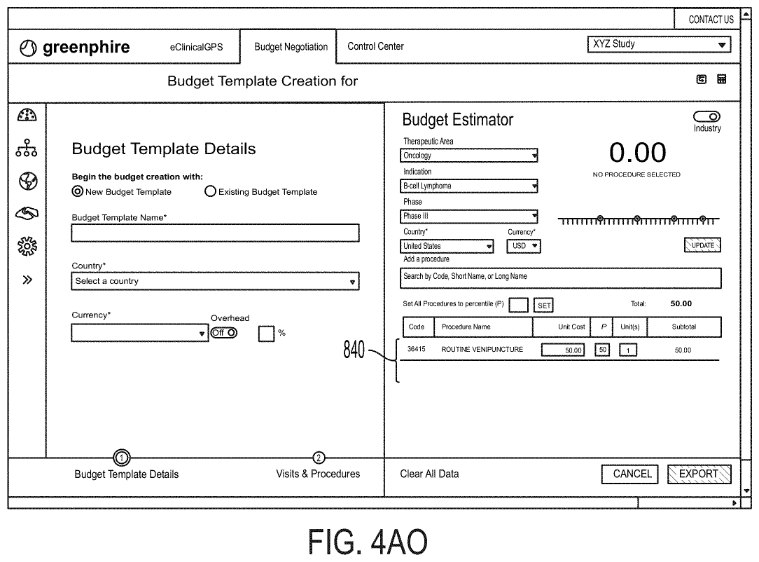

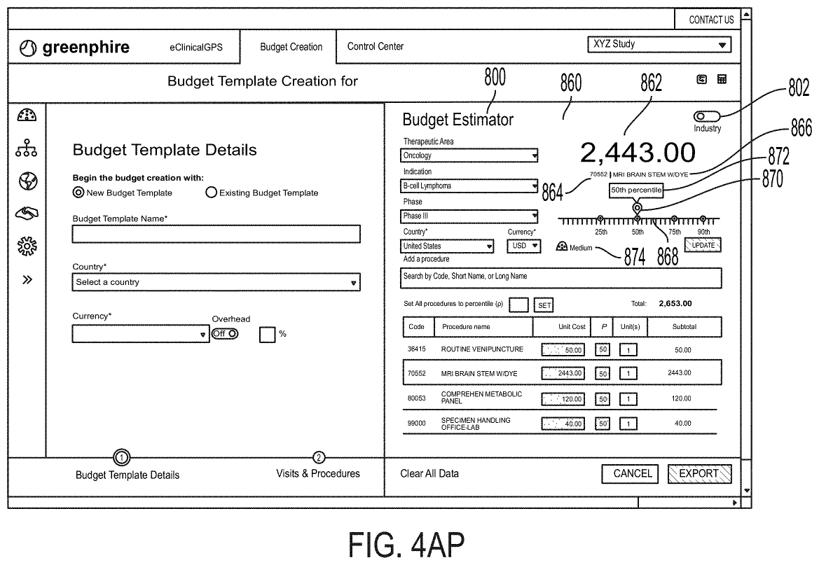

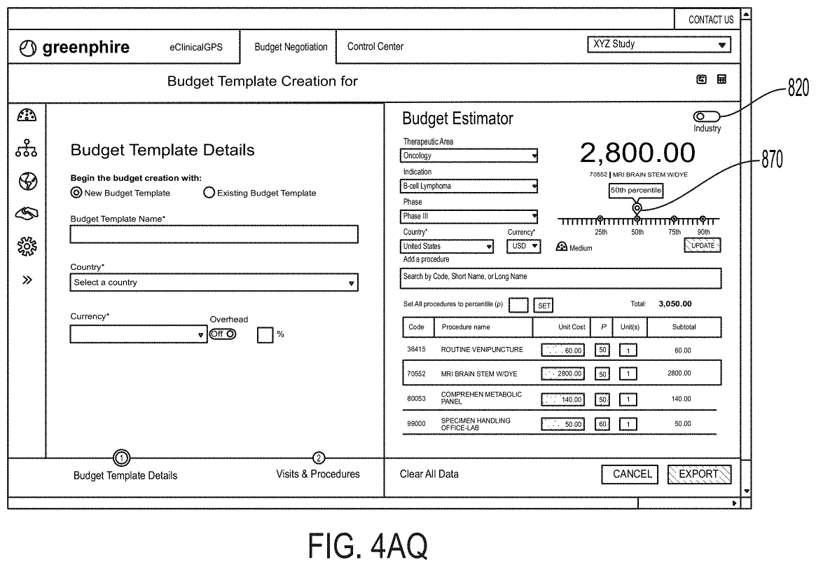

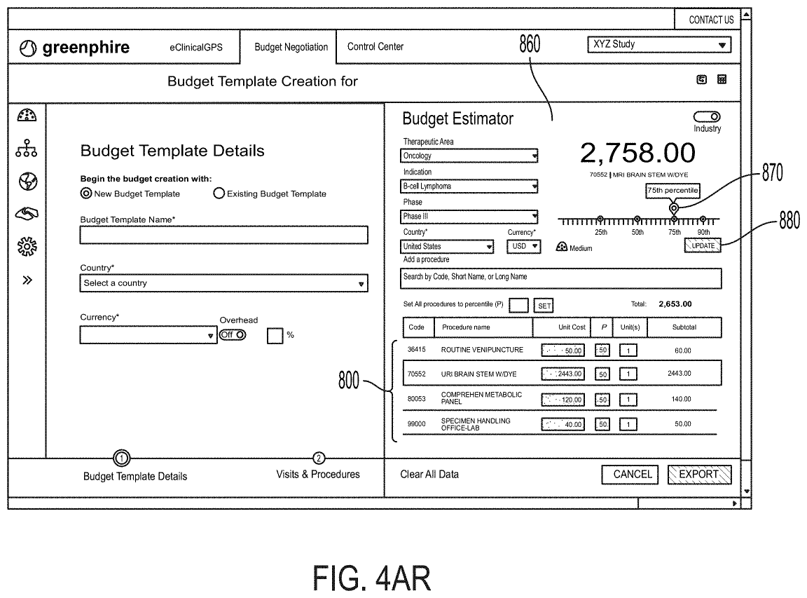

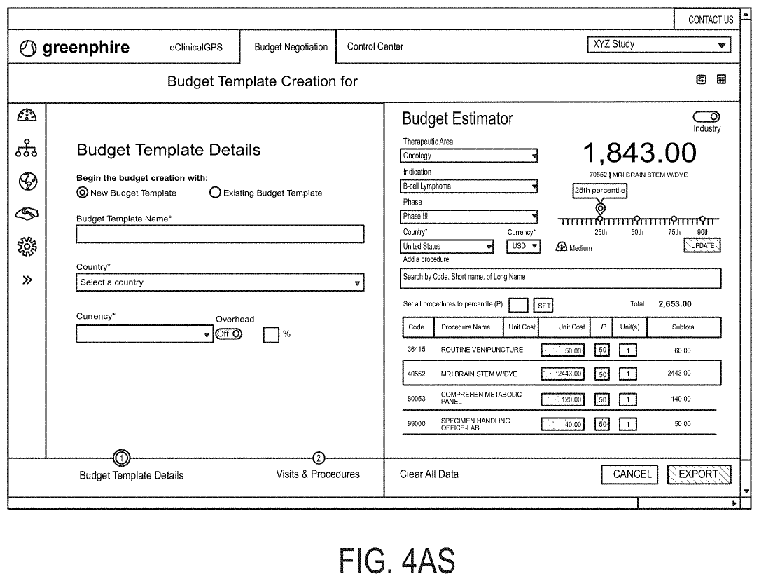

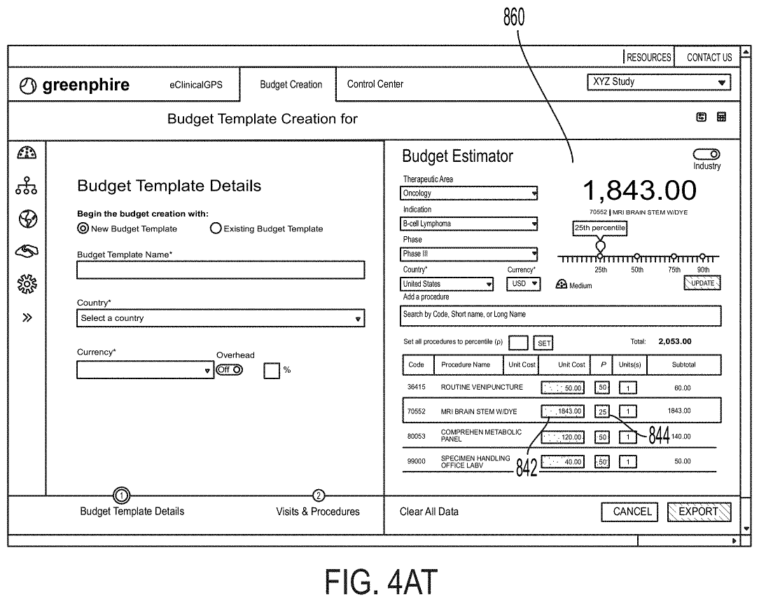

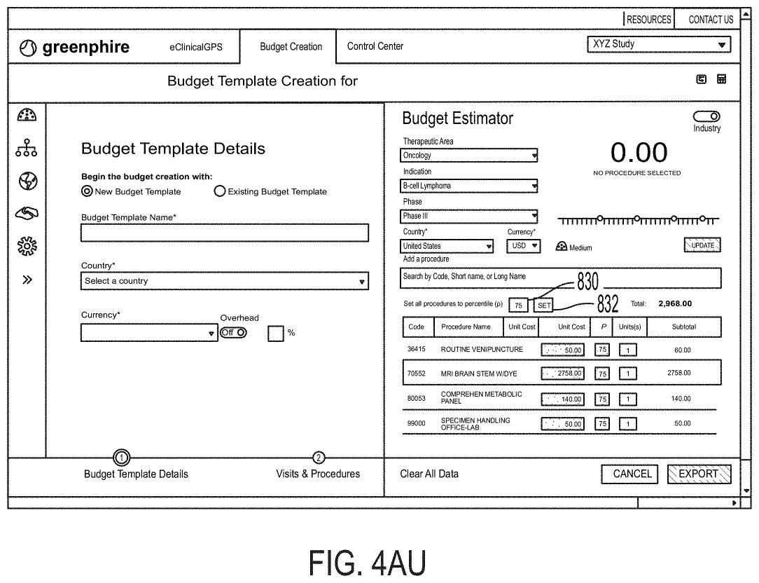

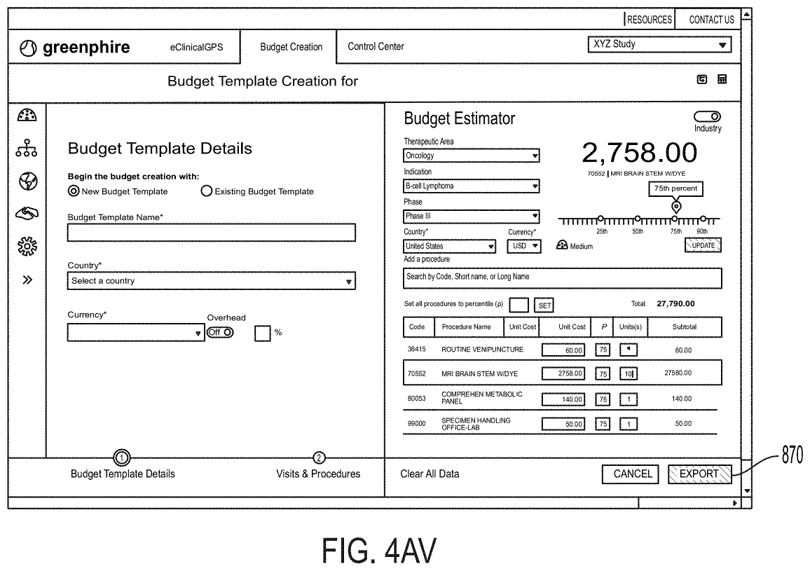

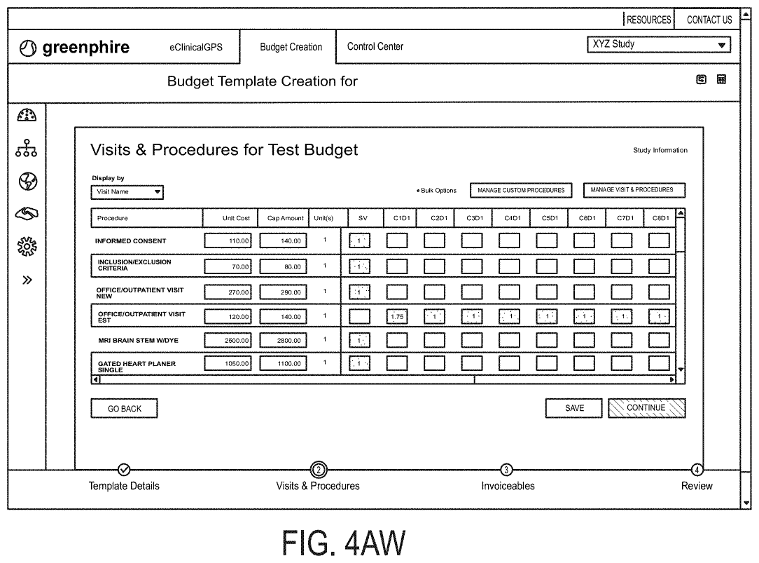

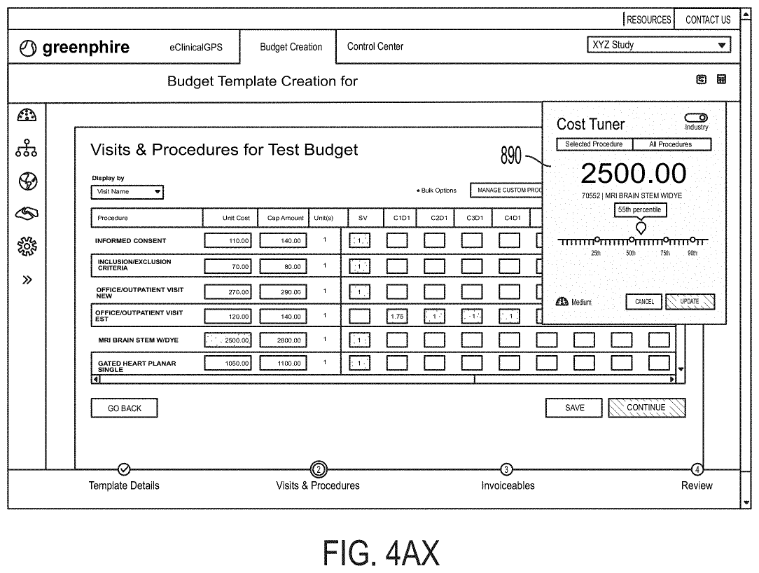

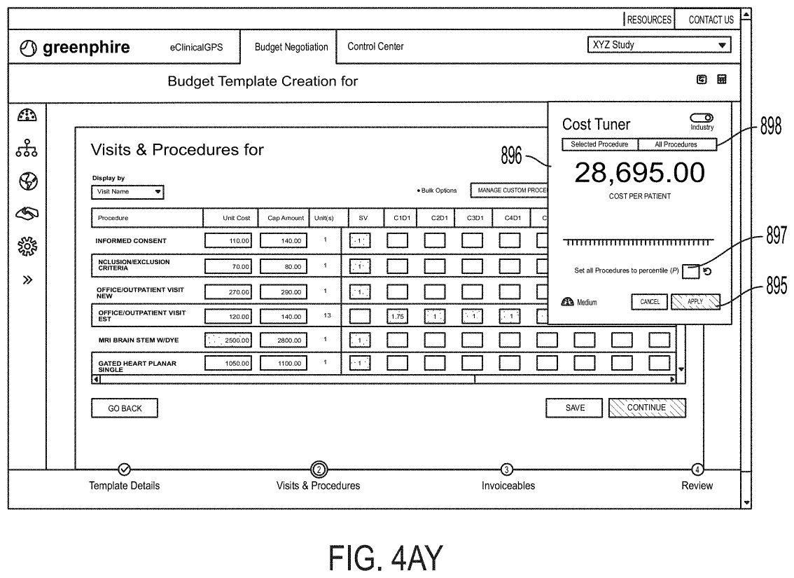

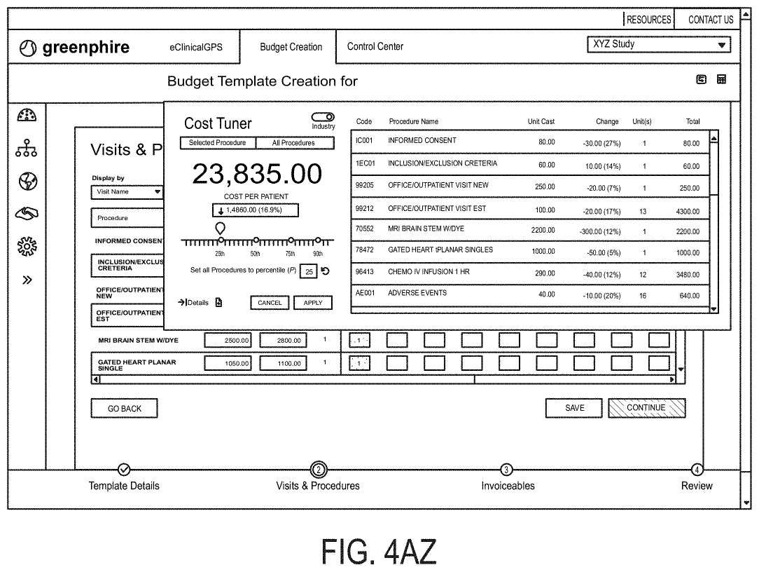

FIG. 3 is a flow diagram illustrating an exemplary method of graphical user interface management (e.g., controlling a display of a computerized system) providing historical data-based forecasting for clinical trials. Additional information relating to the graphical user interface and clinical trial plan building and approval methodology and functionality is provided below with reference to the exemplary graphical user interface windows shown in FIGS. 4A-4AX.

Referring now to FIG. 3, the exemplary method involves storing historical clinical trial payment data for a plurality of clinical trials, as shown at 302. This may include storing historical clinical trial data that includes information broader than just payment information, such as sponsor name, country, supplier names, study names, and any other clinical trial-related data. For example, clinical trial study data identifying non-payment information may be stored as Clinical Trial Study Data 224k in the memory of the CTFS 200, and historical clinical trial payment data 224a (which may include agreements on payments and/or actual payments made) may be stored as Historical Clinical Trial Payment Data 224a in the memory 224 of the CTFS 200, a shown in FIG. 2.

In the example of FIG. 3, the method next involves processing the historical clinical trial payment data to perform an aggregation reflecting a fair market value for a plurality of tasks, namely, those tasks identified in the historical clinical trial payment data and/or clinical trial study data, as shown at 304. As discussed in greater detail below, the aggregation may involve averages (e.g., across sponsors, for a single sponsor, for a single country, etc., for each of a plurality of different clinical trial tasks) or any other statistical or other aggregation to determine a fair market value for a task based on existing data as to what pricing has previously been agreed to, or what payments have been made. In other embodiments, aggregation may be performed in whole or in part at a later point (e.g., on-demand, as needed).

In the example of FIG. 3, the method next involves displaying graphical user interface windows for building a clinical trial study plan including a plurality of clinical trial study tasks, as shown in 306. Building of a clinical trial study plan is discussed below, e.g., with reference to Study Record Creation, Master Template Creation, and Country Template Creation.

### STUDY RECORD CREATION

Referring now to FIG. 4A, a graphical user interface window 400 is shown. This window 400 is displayed at the Sponsor Computing Device 90a by communication of appropriate data from the CTFS 200, in particular, under control of the Study Plan Budder Module (SPBM) 260. As shown in FIG. 4A, the window 400 displays a list 402 of previously created clinical trial studies, which may be retrieved from Clinical Trial Study Data 224k stored in the data store 224, as well as a user selectable button 404 for creating a new clinical trial study after the Create Study button 404 has been selected, in response to which the CTFS 200 is operable to cause display of a study profile panel 401 within the window 400, as shown in FIG. 48. The study profile panel 401 includes fields for receiving and displaying user input for a study name, client name, sponsor name, clinical research organization number, and a specification of an applicable funding account currency, as shown at 408. The information gathered as part of this process may be viewed and edited in a review pan& after selecting a Continue button. After any additional information is gathered in a similar manner, this newly created study (e.g., study name 02242020) is added to the list 402 displayed in the study panel 401 of the window 400, as shown in FIG. 4B. This list of studies and associated graphical user interface display windows may be accessed via a Studies tab 403 of the window 400 as shown in FIGS. 4A and 4C.

### Master Template Creation

Referring again to FIG. 4C, user interface window 400 also includes a Master Template navigation tab 430, which can be used to create a Master Template that effectively defines a template plan for a clinical trial study, including a patient visit schedule, an indication of medical/other procedures (tasks) to be performed at each patient visit, a schedule of invoiceable services/tasks to be performed during the study, etc. Upon selection of the Master Template tab 430, the SPBM causes display of a Master Template Configuration pan& 432 within the window 400. This panel 432 allows a sponsor, using the Sponsor Computing Device 90a, to develop a clinical trial study protocol by developing a desired schedule of patient visits and procedures, etc., using the graphical user interface caused to be displayed by the SPBM 260. To begin doing so, a user may interact directly with a table displayed in subpanel 436. More particularly, the subpanel 436 includes a data entry field 437 for adding a visit to a schedule of visits for the study. As noted from FIGS. 4C and 4D, the data entry field 437 allows a user to provide textual input to name the visit. In this example, as shown In FIG. 4D, the first added visit has the name Screening, and the Screening text 438 is displayed in the field 437. As the user begin selects and/or provides text input into the field, the SPBM 260 causes a next data entry field 437a to be displayed adjacent the former data entry field 437, with the text Add Visit displayed. In turn, this next data entry field 437 is user-selected and configurable with text for naming a next visit in a similar fashion. FIG. 4E shows that a next visit was named Cycle 1 Day 1, and the corresponding text 439 is displayed in the next data entry field 437a. This effectively sets up a tabular display in which cells are defined in rows and columns, and each column corresponds to a particular visit having a name defined in one of the data entry fields 437 so defined. Additional visits have been defined and named in the manner described above, and thus a visit schedule for a clinical trial study (e.g., including a screening visit, a single cycle of 5 visits, and an end-of-treatment visit, may be defined.

In this manner, the SPBM 260 causes display of a graphical user interface that can be used to build/define a visit schedule as part of a plan for a prospective clinical trial study.

Procedures may be added to the clinical trial study plan and associated with visits of the visit schedule via this same panel 432/interface, For this purpose, the subpanel 436 includes a data entry field 440 for adding procedures. More particularly, the displayed data entry field 440 allows the user to provide input to be used to search for and select a medical procedure to be associated with the study. As used herein, the term medical procedure includes actual common medical procedures, such as those readily identifiable in the industry by the American Medical Association's Clinical Procedure Terminology (CPT) codes, as well as other tasks or non-procedures that do not directly correspond to the well-established CPT codes, such as obtaining informed consent as captured in an informed consent form, or performing other tasks common and/or useful for the performance of clinical trials. In this exemplary embodiment, the CTFS 200 is configured to receive and search a data store of CPT codes and associated medical procedures in response to input into the data entry field 440, as shown in FIG. 4F. In this embodiment. CPT (and other procedure) code data is stored in the data stored 24 as Procedure Code Data 224g. It will be appreciated that in other embodiments, the CPT code data may be stored outside the CTFS 200 and accessed via the network 50 on an as needed basis.

Additionally, custom non-CPT procedure codes and associated procedure descriptions (e.g., such as for an informed consent process) may be created (e.g., by a sponsor) and stored in the data store 224 as part of the Custom Code Data 224h. As can be seen in FIG. 4G, data entry of 9900 into the data entry field 452 results in the SPBM's 260 search of the Procedure Code Data 224g, and display of matching codes/procedures in a list 454 associated with the data entry field 452.

A desired procedure can be added to the tabular display by clicking/selecting the procedure in the list of matching procedures. This adds the selected procedure to a tabular display in a table of procedures for the study displayed in the subpanel 436, as shown in FIG. 4H. This results of display of associated text in the data entry field 240, display of any associated code text in an associated Code field 442 in the same row of the table, and display of a next data entry/search field 440a in a next row, beneath the preceding row. This can be repeated to add additional procedures to the tabular display in the subpanel 436. This effectively sets up a tabular display in which cells are defined in rows and columns, and each row corresponds to a particular procedure having a name defined in one of the data entry fields 440 so defined. FIG. 4I shows that additional procedures have been identified in the manner described above and added to the clinical trial study.

Initially, each procedure is added to the tabular display in the subpanel 436 in the order in which they are added. However, listed procedures may be moved and reordered within the tabular display in the subpanel, e.g., by clicking and dragging each procedure via its associated “handle” tab 444, as shown in FIG. 4I. For example, the last-listed procedure with code 99202 in may be moved to be the first-listed procedure in the table. Additional moving, and reordering and movement of all other listed procedures may be performed in a similar manner.

Initially, each procedure is added to the tabular display in the subpanel 436 without a quantity, and without association with any particular visits. Accordingly, for example, the units displayed in a Units field 446 associated with each procedure is initially 0, as shown in FIG. 4J.

However, each procedure can be associated with one or more visits via the tabular display in the subpanel 436. More particularly, the tabular display in the subpanel 436 includes data entry fields 448 as cells defined at the intersection of each procedure row and visit column. Accordingly, the user may provide, input to any data entry fields 448 to add one or more units of each procedure (e.g., procedure code 99205) associated with that data entry field, e.g., 448a, to the visit (e.g., Screening Visit) associated with that data entry field 448a, as shown in FIG. 4K. Upon completion of the entry, if any of the data entry field associated with a particular procedure, the Units field 446 is updated to display the total of all units across all visits for that procedure. This may be repeated as desired to complete the definition the visits and procedures schedule, as desired. A completed visits and procedures schedule, shown in tabular form, is shown FIG. 4L.

In response to right-clicking a mouse on any handle/tab 450 of an associated procedure, the SPBM 260 causes display in the subpanel 436 of a menu that includes a Delete option. This option is user-selectable to delete the associated procedure from the table, and thereby, from the definition of the clinical trial plan. Similarly, in response to right-clicking a mouse on any name of a visit, the SPBM 260 causes display of a menu that includes a Delete option. This option is user-selectable to delete the associated visit from the table, and thereby, from the definition of the clinical trial plan.

In a manner similar to that described above, in response to left-clicking any name of a visit, the SPBM 260 causes display in the subpanel 436 of a user-manipulable handle 450 (in the graphical user interface window) that can be selected (clicked) and dragged to move the select column (and visit-related data) to another column location within the table. In response to doing so, the SPBM revises the tabular display to include the moved column in the new location, and to display the other columns moved accordingly. In FIG. 4M, the Screening visit, and associated procedure counts, have been moved from before the Cycle 1 Day 1 visit to after the Cycle 1 Day 1 visit, for illustrative purposes. This effectively allows for refinement of the definition of the clinical trial plan via this tabular interface displayed in the subpanel 436.

In this manner, the SPBM 260 causes display of a graphical user interface that can be used to build/define a list of procedures to be performed as part of a plan for the prospective clinical trial study. The plan may be saved as Proposal Study Data 224c.

After creation of the list, a summary panel 465 may be displayed in the window 400, as shown in FIG. 4N. This provides a recap, sorted by the list of procedures. In certain embodiments, the mapping described below may be performed in another part of the workflow described herein. However, in this example, in a summary panel 465, each procedure and associated visit is initially displayed in associate with a displayed warning icon 469. The presence of the warning icon indicates that the procedure has not yet been mapped to EDC-based reporting from a clinical site, such that reporting of task completion data from the clinical site will be recognized as an indication of completion of the associated visit/procedure, so as to trigger payment to the clinical site for completion of the associated visit procedure. Selection of the Information input icon 471 in FIG. 4N results in display of mapping panel 470, as shown in FIG. 4O.

This mapping panel 470 allows for the procedure, e.g. the MRI Brain Stem procedure of the Screening visit, to be matched to or mapped to the performance of this procedure during the screening visit as captured by data input to an external electronic data capture (EDC) system 505 of the type used by clinical trial sites in recording performance of clinical trial tasks, as shown in FIG. 1. Here, the panel 470 displays reference name data entry field 472, and in this example the user/sponsor has provided user input of 123. This code is known to the sponsor as the result of the sponsor's configuration of and/or knowledge of the operability of the EDC system that will be used by the clinical site, as required by the sponsor. Effectively, this provides a way for the CTFS 200 to receive or transmit data from the external EDC System 505 in a coordinated and meaningful fashion. In this example, the EDC System's communication/reporting of the 123 code will be recognized by the CTFS 200 as indicating completion of the MRI Brain Stem procedure of this Screening visit, so that payment can be authorized by the CTPMS 400 and/or PPS 405 based on data communicated from the CTFS 200 in response to receipt of this information from the EDC system.

After the mapping has been completed, a Completion icon 484 is displayed in the summary review panel to indicate that the mapping has been completed.

To complete the review process and save the clinical trial study plan as so developed, the user may select the finalize configurations button 486 (e.g., after resolving all mappings) displayed in the panel, as shown in FIG. 4P. This will result in storage of this master template configuration in as Master Template Study data 224d in the data store 224 of the CTFS 200, as shown in FIG. 2. This template may subsequently be retrieved and used to develop/define other studies, with a great savings in effort, and with recall of previously clinical trial plan protocol. Additionally, this template is used to develop country (and currency)-specific study templates as described below.

In this embodiment, the SPBM 260 also causes display of a Bulk Upload option selectable to allow for upload to the CTFS 200 of master template data of the type described above in a data file, such that the relevant data can be imported to create a master template within the CTFS 200. By way of example, the data file may be a Microsoft Excel spreadsheet file or other data file. This can allow for creation of budget templates outside of the CTFS system, e.g., within Excel, as an alternative to the process described above. Relatedly, in this embodiment, the SPBM 260 also causes display of an Export option 488 selectable to allow for download from the CTFS 200 master template data as a data file, such that the relevant data can be exported from the CTFS 200, as shown in FIG. 4N. By way of example, the data file may be a Microsoft Excel spreadsheet file or other data file. This can allow for review of budget templates outside of the CTFS system, e.g., within Excel, as an alternative to the process described above.

Alternatively, instead of the bulk upload described above, data can be copied from a source and be pasted directly into the tabular display described above, eliminating the need for a bulk upload described above while achieving a similar effect.

The SPBM 260 also causes display of a Manage Custom Procedures option 489, as shown in FIG. 4Q. This option 489 is selectable to allow for creation and tracking of custom procedures, as referred to above. These custom procedures may identify any task, as desired, and in particular are useful for procedures that are not typical medical procedures associated with a CPT code. Selecting this option causes display of a Manage Custom Procedures panel 500, as shown in FIG. 4Q. When this panel is accessed, a list of custom procedures previously defined, and stored in the CTFS 200 as Custom Code Data 224h of the Procedure Code Data 224g is displayed in a list 502 within the panel 500, as shown in FIG. 4R. A new procedure can be created and added to the list by selection of the Add A Custom Procedure icon 504 displayed in the panel 500. Selection of this icon 504 results in display of and associated panel 506 displaying text entry fields 508, 510 allowing for input of a new procedure code by which this new custom procedure will be tracked, and a corresponding procedure name description, as shown in FIG. 4R. Selecting the OK button 510 results and adding of the new procedure to be list 512. In this manner, a sponsor can define a new procedure that may be added to a clinical trial plan presently being defined, and this new procedure will be retained by the CTFS 200 so that it can be selected rather than defined and added to a new clinical trial plan in the future. This functionality may also be provided at the Master Template level.

In certain embodiments, the treatment arm creation/ordering, etc. described below may be managed via a tabular display similar to that described above. In the example shown, returning to the Master Template Configuration panel, as shown in FIG. 4S, this panel also includes a list from which a user may select a treatment arm, as shown at 520. Selecting a treatment arm from this list displays the treatment arm panel 522 which includes a list 524 of stored treatment arms. Additionally, this panel 522 includes a user selectable Create A Treatment Arm button 526 for creating a new treatment arm. Definition of multiple treatment arms allows for definition of similar of related sets of tasks while also allowing for differentiation across treatment arms. Additionally, the system allows for one treatment arm to serve as the starting point/basis for a next treatment arm, and then to allow editing. By way of example, two treatment arms may be defined for use with patients, but one may be male-specific and exclude a requirement for a pregnancy test, and the other may be female-specific and include a requirement for a pregnancy test. In this manner, the system allows for quickly, easily and accurately creating similar but different treatment arms, and associated clinical trial plans and associated budgets. These treatments arms are tracked on a per study basis, and are stored as part of the master template study data 224d for each master template configuration. Selecting the Create A Treatment Arm button 526 results in display of a create a new treatment arm panel 528 that includes a dialog boxes 530, 532 allowing a user to enter a name and description of the new treatment arm, and a menu 534 allowing the user to specify the relative position of the treatment arm, as shown in FIGS. 4T and 4U. The relative position sets the display order of the treatment arms. Thus, a position of 2 will show that treatment arm second in relative order. After providing this input, selection of the OK button results in adding of the treatment arm to a list 538 in order corresponding to the relative position specified, as will be appreciated from FIGS. 4V and 4W. Additional treatment arms may be added in a similar fashion. Subsequently, a treatment arm may be selected from a list of treatment arms in the Master Template Configuration panel and selection of a particular treatment arm will result in display of a list of procedures associated with that treatment arm, as will be appreciated from FIG. 4X.

As will be appreciated from FIG. 4Y, a particular treatment arm, e.g. treatment arm B in this example, may be selected and by selecting the manage visits and procedures button, the manager visits and procedures panel is displayed for treatment arm be at which point additional procedures may be added to only this particular treatment arm, in a manner similar to that described above. Visits and procedures can also be deleted, e.g., by selecting an icon about a column to delete a visit, or by right-clicking on the table and selected a “Delete Row” option. The revised list of procedures associated with that treatment arm is then displayed in the list of procedures in the master template configuration panel, as shown in FIG. 4Z. Other treatment arms may be reviewed and modified in a similar manner.

Referring now to FIG. 4AA, the Master Template Configuration panel 432 further includes an Invoiceables tab 550 for defining invoiceable deliverables for clinical trial study. Examples of invoiceables include blood draw services, blood lab tests, chemotherapy infusions and other items that are approved, and for which an associated payment has been negotiated, for a particular clinical trial study, but which are not required in the Visits and Procedures list, but rather are requestable/performable on an as-needed basis by the clinical trial site. For example, if a pregnancy test is not included in a Visits and Procedures list (e.g., in a Treatment Arm for a Visits and Procedures list), the pregnancy test may be included as an Invoiceable, and may simply be performed and invoiced on ad hoc basis, as needed. When this tab 550 is selected, a subpanel 552 is displayed, as shown in FIG. 4AA. This subpanel 552 includes a dialog box 556 for receiving user input that can be used to search for an invoiceable by code (which may be a CPT code or a custom defined/specified code within the CTFS system, similar to the procedure codes described above) or by name/description. These codes and associated invoiceable descriptions may be stored as part of the Procedure Code Data 224g in the data store 224 of the CTFS 200. FIG. 4AA displays a list 560 of invoiceables descriptions and associated codes retrieved from the data store 224, and matching the search input provided in dialog box 556. This subpanel 552 further includes a dialog box 568, displayed in the same row, for receiving user input identifying a quantity of the associated invoice.

FIG. 4AB displays a list 562 of invoiceables (and associated units and procedure codes) added to this particular Master Template Configuration so that invoiceables may be reviewed in list format. This subpanel, like the subpanel 436 described above, also include tabs and handles that are user clickable and draggable/selectable to permit movement and reordering of the listed invoiceables. In this manner, the sponsor has defined a clinical trial plan including a visit schedule, a desired list of procedures for each visit, and a list of invoiceable for the study, and that information is captured in the Master Template for the study, created as described above. This Master Template is stored and available to be copied and used in modified for unmodified form to create a new study. Additionally, this information captured in the Master Template is used as the starting point for creating country-specific and supplier-specific clinical trial plans (resulting in supplier-specific clinical trial agreements) for the same study, as minor variations across individual suppliers for a single clinical trial study is not uncommon, and is often necessary as a practical matter. Additionally, the information captured in the master Template is used by the CTFS and the sponsor for negotiating payments amounts for those items with suppliers, as described below.

### Country Template Creation

As shown in FIG. 4AC, the window 400 further includes a Country Template tab template 600, which can be used to create clinical trial study cost forecasts for the clinical trial study plan (as defined), on a per-country and/or on a per-country/per-currency basis. Selection of the Country Template tab 600 will result in display of a budget template panel which displays a list of stored country/currency-specific budget templates for the defined clinical trial study plan associated with this particular clinical trial study, if available. This panel further includes a Create Template button for creating a new country/currency budget template that includes a budget based upon payments for deliverables under the corresponding clinical trial study.

In response to section of the Create Template button, a Budget Template Details panel 610 is displayed in the window 400. This panel 610 includes data entry fields 612, 614, 616 for receiving user input identify a budget template name, country, and currency, respectively, as shown in FIG. 4AC. Additionally, this exemplary panel 610 includes a user-manipulable element 618 operable to turn on or off Overhead functionality, as well as a data entry field 620 for specific an overhead to be applied as a percentage of other costs. When the Overhead functionality is on, pricing may be increased by the specified percentage in order to account for general facility overhead in pricing negotiations. Alternatively, the corresponding portion of the overhead may be included, but may be tracked separately from the remainder of the task pricing, so that the country/clinical trial site/supplier overhead portion is tracked separately from and/or excluded from the fair market value calculations for the associated tasks. Additionally, the overhead may be tracked separately to all for a compilation of estimated and/or fair market value overhead values on a per country, clinical trial site and/or supplier basis, e.g., for budget calculation purposes and/or for clinical trial plan building purposes. After the user has provided the appropriate input, the user may select a Continue button 622 displayed in the panel.

Upon selection of the Continue button 622, the SPBM 260 causes display of a Visits And Procedures panel 624, which includes a list 626 of codes, procedures and an aggregate count of units of each procedure for a particular treatment arm displayed as shown in FIG. 4AD. It should be noted that this list form is provided by way of example as an alternative embodiment to the tabular form described above with respect to the Master Template. The tabular form described above would be replaced with the list form described here, in certain embodiments. Similarly, the list form described here, could be replaced by the tabular form described above, in certain embodiments.

Referring again to FIG. 4AD, this information is inherited/copied from the corresponding Master Template, and thus this information in the Country Template is the same as in the corresponding Master Template, at least initially, although the information may be modified for this particular Country Template if desired, which will not result in corresponding changes to the corresponding Master Template. Thus, one or more per-country (and/or per-currency) templates may be created quickly and efficiency, by leveraging the prior creation of the study's Master Template, and making changes as needed/desired on a per country (and/or per-currency) basis. The Country Templates created are stored in the CTFS, and may subsequently be retrieved from memory and used to create new Country Templates for the same or other studies, which provides additional efficiencies and accuracy to the sponsor, and serves as a “knowledge base”/repository of historical study information.

Referring again to FIG. 4AD, in association with each procedure, a user-selectable button (e.g. a plus sign) 628 is included to expand a record for the associated procedure. FIG. 4AE displays a list 630 of visits associated with the corresponding procedure in response to selection of the expand button 628 for the ROUTINE VENIPUNCTURE procedure. This list of procedures and visits corresponds to that created as described above. Pricing proposals may then be added to the Country budget template for each task, and may be stored as Proposal Study Data 224c.

In this exemplary embodiment of FIG. 4AC et seq. and particularly FIGS. 4AE-4AF, pricing proposals (for any procedures/invoiceables/tasks) may be entered in straightforward fashion, e.g., by the sponsor simply identifying payments amounts, as discussed below. In an alternative embodiment, the SPBM 260 works in concert with the Fair Market Value Engine (FMVE) 240, and/or uses Fair Market Value Data 224b created by the FMVE, to cause display of fair market value data for various tasks and procedures, based on historical payment data 224a reflecting actual payments made for the same or similar tasks, to inform the user and/or guide the user in determining pricing proposals for the tasks/deliverables of the Country budget template, as discussed below.

Referring again to the FIG. 4AE, the panel 624 includes a display of data entry fields 632 for receiving user input of an offer amount, namely, an amount that the sponsor would like to offer to pay for the associated procedure at each visit. Further, the panel 624 includes a display of data entry fields 634 for receiving user input of a Cap Amount, namely, an amount that the sponsor does not wish to exceed for payment for the associated procedure at each visit. Further still, this panel 624 includes a display of data entry fields 636 for receiving user input of a number of units of the corresponding procedure at each visit. Further still, this panel 624 includes a display of the aggregate total of units entered into data entry fields 636 on a per procedure basis, as shown at 638 in FIG. 4AE. FIG. 4AF shows exemplary input provided in data entry fields 632, 634, and 636 for various procedures, for illustrative purposes, as well as an aggregate total for the ROUTINE VENIPUNCTURE procedure at 638. Notably, this is for Treatment Arm A, as shown in FIG. 4AF, and an alternative treatment arm, such as Treatment Arm B, may be selected via the panel, and similar information may be provided in the similar manner, although notably the data entered into the data entry fields 632, 634, 636 may vary from treatment arm to treatment arm.

Selecting the “Continue” button leads to display of an Invoiceables panel 650 within the window 400. The invoice of panel 650 displays a list 652 of invoiceable tasks by name and code. This list of invoiceables corresponds to that created as described above. However, in this workflow, the panel 650 includes a display of data entry fields 654 for receiving user input of an offer amount, namely, an amount that the sponsor would like to offer to pay for the associated invoiceable. Further, the panel 650 includes a display of data entry fields 656 for receiving user input of a Cap Amount, namely, an amount that the sponsor does not wish to exceed for payment for the associated invoiceable. Further still, this panel 650 includes a display of data entry fields 658 for receiving user input of a number of units of the corresponding invoiceable.

This panel 650 also includes a Continue button 660. Upon selection of this button 660 a Template Overview panel 690 is displayed in the window 400, as shown in FIG. 4AG. This panel 650 includes a summary that displays a mapping of procedures to visits and indicates costs for each procedure at each visit, as shown in FIG. 4AG. In particular, a list of procedure names is provided in rows of a table 692 displayed in the panel 690 and each visit is assigned to a separate column in the table 692, and the cost for each procedure at each visit is shown at the intersection of each row and column where applicable. Thus, the template overview panel 690 provides a particularly compact and visually meaningful display of study plan information in tabular form. Such a compact display is particularly helpful for clinical trial planning and budgeting purposes. Further, this panel 690 may display one such summary for each treatment arm, and may further include a similar tabular form summary for the invoiceables defined as part of this clinical trial study, as shown in FIG. 4AH.

The system may again display the country/budget templates panel for this study, with the newly created budget template (in this case, specifically for the United States) added to the list 696 of stored budget templates, as shown in FIG. 4AI. This country-based template is stored in the data store 224 of the CTFS 200 as country template study data 224e, as shown in FIG. 2. Additional templates, e.g., for other countries, can be created and stored in a similar manner for this particular clinical trial study. In this manner, costs can be specified and negotiated on a country by country basis for each clinical trial study. Display of all of these windows and the receipt and storage of information is managed by the study plan builder module 260 of the UIDE 250 of the CTFS 200. Additional studies can be defined and stored, additional master templates for those studies can be designed and stored, and additional budget templates may be defined and stored in a manner similar to that described above.

In this manner, the SPBM 260 causes display of a graphical user interface that can be used to build/define a list of invoiceables to be performed as part of a plan for the prospective clinical trial study.

In this manner, the sponsor has defined a country/currency-specific clinical trial plan including a visit schedule, a desired list of procedures for each visit, a list of invoiceables for the study, and associated proposed and capped payment amounts for each of those items. Notably, multiple templates may be created, each with its own unique variations if desired, for example for different currencies in the same country, and/or for different countries. All such information is captured in each Country Template for the study, created as described above. This Country Template is stored and available to be copied and used in modified for unmodified form to create a new country template. Additionally, this information captured in the Country Template is used as the starting point for creating a supplier-specific clinical trial plans and for negotiating payment amounts for those items with each supplier (ultimately resulting in supplier-specific clinical trial agreements) for the study, as described below.

Accordingly, as described above, the method involves displaying graphical user interface windows for adding a first/next task to be included in the clinical trial study plan, as shown at 308 in FIG. 3. If a first/next task is to be added, the first/next task is added to the clinical trial study plan, as shown at 308 and 310, and then the system retrieves and displays fair market value data relating to the first/next task, as shown at 312. The user, after/while viewing such fair market value for the task, may then assign a payment offer for that task, as shown at 314. It is then determined if the building of the clinical trial plan is finished, as shown at 316, and if not, a next task may be added, etc., as shown at 316 and 308. This continues until the plan is finished. It should be appreciated that in the illustrative example of FIG. 3, the fair market value is displayed after each task is added, but in other embodiments, the fair market value may be displayed only after all (or multiple) tasks have been added to the clinical trial study plan. Further, it should be appreciated that in the example described herein, the development of master-, country- and/or supplier-specific templates are developed for building study plans, which is advantageously efficient for creating of new study plans, or proposed study plans, based on previously-created study plans, and/or in performing “what if”-type analysis, e.g., to forecast costs of a hypothetical study in Country 1 (or with Supplier 1) and in Country 2 (or for Supplier 2), and then to compare such costs in selecting a country, negotiating payment/task terms with one or more suppliers, finalizing a clinical trial strategy, etc. However, in other embodiments, such master-, country- and/or supplier-specific templates may not be used. Rather, a one or more discrete study plans may be developed separately. Additional information in relation to the fair market value data generation and analysis provided below.

### Fair Market Value Analysis

As referred to above, in an alternative embodiment, the SPBM and/or FMVE 260 displays graphical user interface windows during development of the Country template that provide guidance to the sponsor user in determining pricing proposals for the tasks/deliverables of the Country budget template, as discussed below with reference to FIGS. 4AL-4AX. In one embodiment, the FMVE 240 pre-processes the historical clinical trial payment data 224a to create and store fair market value data 224b in the data store 224. Such fair market value data 224b is then subsequently available to the SPBM 260 for display of graphical user interface windows providing fair market value data for use in developing the Country budget template. In an alternative embodiment, the SPBM 260 works in concert with the Fair Market Value Engine (FMVE) 240 during the development of the Country template to cause display of fair market value data and associated graphical user interface windows providing fair market value data for use in the developing the Country budget template and/or in creating and displaying clinical trial budget forecast information.

Accordingly, in this alternative embodiment, selection of the Continue button 622 of FIG. 4AC results in display of Fair Market Value Estimator panel 800, as shown in FIG. 4AN. This panel may be used to develop a list of procedures in a manner similar to that described above, including searching for procedure codes, adding procedures and procedures codes to a list, etc., in a manner similar to that describe above, as will be appreciated from FIGS. 4AN-4AP. Additionally, however, this panel 800 also provide data input fields for filtering and displaying fair market value data. For example, this panel includes data entry fields 802, 804, 806,808, 810 for filtering fair market value data (for costs of associated procedures) by Therapeutic Area, Indication, clinical trial study Phase, Country and Currency, as shown in FIG. 4AN. Additionally, a user-manipulable element 820 (e.g., slider) is displayed in this panel to allow for filtering and use of industry data (including the same sponsor's data and data of other sponsors), as shown in FIG. 4AN, or to allow for filtering and use of client data (including only the same sponsor's data), as shown in FIG. 4AQ. Additionally, this panel includes a data entry field 830 for filtering and use of fair market value data based on a percentile (e.g., 25^{th}, 50^{th} or 75^{th} percentile data), and a Set button 832 to apply to specified percentile filter. All of these filters, in combination, govern which fair market value data will be used to display fair market value data in the panel 800.

FIG. 4AO illustrates the addition of a ROUTINE VENIPUNCTURE procedure to a list 840 of procedures. The procedure is displayed in the list 840 in association with fields 842, 844, 846, 848 for displaying and/or entering unit cost, fair market value data percentile for the cost, and associated units, as well as a subtotal for that procedure, as will be appreciated from FIG. 4AM. This can be repeated for other procedures added to the list 740. In this case, when no percentile value is initially specified, values for a default percentile are displayed, namely, 50% in this example. This may be user-configurable on a per-study or per-sponsor basis. There is also a field 750 for displaying a total of all costs. This is helpful for clinical trial budget planning/forecasting purposes.

As shown in FIG. 4AP, any procedure in the list may be selected for additional fair market value analysis and detail. As shown in FIG. 4AP, selection of a procedure results in display in an FMV detail panel 860 of the Budget Estimator panel 800 of additional fair market value data. In particular, this display includes fields displaying the procedure cost 862 in the selected currency, the (CPT or other) procedure code 864, procedure description 866, a display of a percentile range 868 and a marker 870 showing the fair market value data percentile of associated/displayed procedure cost, a textual display of the percentile 872, and a gauge 874 in the nature of a qualitative display indicating whether the cost is low, medium, or high, based on the selected percentile.

For forecasting, budget planning, and historical data-based analysis, the Industry/Client data source filter's manipulable element 820 may be toggled (in this case, from Industry to Client), to provide further information about costs relative to fair market value, as will be appreciated from FIGS. 4AP and 4AQ. Similarly, the marker 870 may be manipulated (e.g., clicked and dragged) to select a different FMV data value corresponding to a different percentile, in response to which the display in the FMV detail panel 860 is responsively updated, as will be appreciated from FIGS. 4AR and 4AS. Notably, the price proposal data is not yet changed in the procedure list 840. This allows for performance of “what if” or scenario analysis within the FMV detail panel 860, e.g. by direct manipulation of the marker, before updating the budget in the list 840. After the sponsor user is satisfied with the price point relative to the fair market value data, the Update button 880 displayed in the FMV detail panel 860 may be selected to update the values displayed in the cost and percentile fields 842, 844 for the associated procedure in the list 840, as shown in FIG. 4AT. Clicking/selecting the same procedure in the list 840 again will deselect the associated procedure and clear the display in the FMV panel 860. This may be repeated as desired from the other procedures in the list.

Additionally, the Budget Estimator panel 800 includes the bulk data entry field 830 for filtering and use of fair market value data based on a percentile (e.g., 25^{th}, 50^{th}, or 75^{th} percentile data), and a Set button 832 to apply to specified percentile filter. As will be appreciated from FIG. 4AU, specifying a percentage in field 830 and selecting Set will set the percentile for all procedures concurrently, and will result in display of corresponding percentile data values for Unit Cost for all listed procedures. The subtotal and total are automatically updated accordingly. Changing the number of Unit in the units field for any procedure results in automatic updating of the subtotal and total values displayed in the panel, as will be appreciated from FIG. 4AV. Selecting the Export button 870 displayed in the panel 800 will finalize this process and result in export of data as a spreadsheet (e.g., Microsoft Excel) or image (e.g., PDF) file and/or display of an associated budget in tabular form, as shown in FIG. 4AW. In this tabular display, the final selections of the unit cost for each procedure is displayed (and is editable). Additionally, Cap Amounts may be added by the sponsor user, as described above. The tabular display shows procedures in rows, associated unit cost and Cap Amount, as well as the total number of units of each item included in the budget, and a breakdown showing the number of units at each visit for all visits in the defined visit schedule. This tabular view is a particularly compact and effectively display of information.

Additionally, any procedure displayed in the table may be selected to cause launch of a Cost Tuner panel 890 which includes displays and functionality similar to that of the Budget Detail 860 panel described above, as will be appreciated from FIG. 4AX. The amount displayed in the table will not be updated unless the Update button 894 is selected. The Cost Tuner panel further includes user-selectable options 896, 898 for performing the cost tuning/review/forecasting analysis on a per procedure basis (as shown in FIG. 4AX), or for all procedures in aggregation (as shown in FIG. 4AY). When the All Procedures option 898 is selected, the Cost Tuner panel 890 includes a data entry field 897 for receiving input of a percentile selection to be used for retrieval and use of fair market value data across all procedures. Selecting the Details button 897 (displayed after entry of a percentile value) results in display in the Cost Tuner panel 890 of a list of all procedures and associated codes, unit costs, change relative to prior value, number of units and total cost on a per-procedure basis, as shown in FIG. 4AZ. None of the values are updated in the tabular display of costs unless the Apply button 895 is selected in the Cost Tuner panel 890.

All of this functionality is provided similarly in corresponding fashion for invoiceables, as well as for visits and procedures. In certain embodiments, the Budget Estimator panel and functionality is also accessible outside of the workflow described herein, so that the user need to define a study, master template, etc. before accessing the Budget Estimate, so that a sponsor user may review fair market value data at any time, even outside of the workflow described herein for building a clinical trial study. Accordingly, robust “what if” and scenario analysis, based on historical clinical trial payment data, and determinations of fair market value payments for such tasks, either sponsor-specific or industry-specific, may be performed to guide the sponsor in determining budgets, payment offer amounts, payment cap amounts, or otherwise supporting the sponsor's negotiation with suppliers to reach agreement as to amounts to be paid for various deliverables under an agreement with the supplier. The CTFS acts as a knowledge base, allowing a sponsor to readily access and leverage not only its own past experience in terms of suitable payments, but also that of other sponsors, which can be useful in avoiding unnecessary overpayment or underpayment for various clinical trial deliverable tasks.

### Supplier-Specific Template

After the clinical trial study plan has been defined (e.g., as described above and as discussed with reference to 302-316 of FIG. 3, the CTFS may be used to negotiate payment/task terms with one or more suppliers, as referred to above. Accordingly, the method next involves the CTFS 200 causing display of graphical user interface windows at at least one supplier device, for review of the clinical trial study plan and task payment offers developed as described above, as shown at 318 of FIG. 3.

Accordingly, as shown in FIG. 4AJ, the window 400 further includes a Supplier-Specific Negotiations tab 700, which can be used to create a supplier-specific cost forecast for the clinical trial study plan (as defined), and to negotiate payments for items included in the clinical trial study plan. Referring now to FIG. 4AJ, selection of the negotiations tab 700 results in display of the Negotiation Details panel 702. This functionality and related functionality are performed and/or managed by the Study Approval Module (SAM) 270 of the UIDE 250 shown in FIG. 2. Referring again to FIG. 4AJ, panel 702 includes data entry fields 704,706 for entering a negotiation name, and selection of a country/budget template created as described above and displayed in a list retrieved from the data store 224 of the CTFS 200. Panel 702 also includes a data entry field 708 for specifying a currency 710 for the price negotiation, a user-manipulable element 710 providing an indication as to whether or not to include an overhead expense allocation, and a data entry field 712 for receiving user input as to a percentage to be applied as the overhead expense allocation, as shown in FIG. 4AJ.

Panel 702 further includes a data entry field 714 configured as a drop-down list allowing a sponsor user to select a site name from a list of clinical trial site names stored as Clinical Trial Site data 224i stored in the data store 224 of the CTFS 200. Panel 702 further includes a data entry field 716 configured as a drop-down list allowing a sponsor user to select a payee name from a list of clinical trial site names stored as clinical trial site name data 224i stored in the data store 224 of the CTFS 200. For example, outside of the U.S., there are typically multiple parties or payees that could be paid that are part of/associated with a clinical trial site, such as the principal investigator (PI) himself/herself, members of the study team such as nurses or other providers, or even specific departments within a site (i.e. radiology). So for every site, there could be multiple entities associated with that site that could receive payment. Notably, the payee names, clinical trial site names, and associated contact person name, email address, associated banking data, etc. may be referenced as data stored in the CTPMS 305 (as part of the normal payment management process of the CTPMS 305) and/or may be stored in the CTFS 200 as Clinical Trial Site data 224i (e.g., as retrieved from the CTPMS 305) for the uses described herein. This provides some continuity, and ease of reference to the sponsor in identifying and working with clinical trial sites, etc. that the sponsor has worked with in the past. Additionally, graphical user interface functionality is provided to allow for adding of new payee names and/or clinical trial site information if desired, which is then stored in the CTFS as Clinical Trial Site data 224i for future reference and use with the CTFS (and CTPMS) system.

Panel 702 also includes data entry fields 718, 720, 722 for identifying a desired number of screens subjects a target failure rate and a target dropout rate, this data is used to calculate and display an estimated number of enrolled patients as shown in subpanel 724 and an estimated number of completing subjects as shown in subpanel 726 of FIG. 4AJ. After the required information has been provided, a Continue button 730 is displayed within the panel 702.

Selection of the Continue button 730 results in display of a Visits And Procedures panel 732, in which a similar list of procedures names and the associated codes, Offered payment amount, Cap Amount and Units are displayed on a user selectable, per treatment arm basis. This information is inherited/copied from the corresponding Country Template, and thus this information in the Supplier/Negotiations Template is the same as in the corresponding Country Template, at least initially, although the information may be modified for this particular Supplier Template if desired, which will not result in corresponding changes to the corresponding Country Template. Thus, one or more per-supplier templates may be created quickly and efficiently, by leveraging the prior creation of the study's Country Template, and making changes as needed/desired on a per-supplier (and/or per-currency) basis. The Supplier Templates created are stored in the CTFS, and may subsequently be retrieved from memory and used to create new Supplier Templates for the same or other studies, which provides additional efficiencies and accuracy to the sponsor, and serves as a “knowledge base”/repository of historical study information. Accordingly, these entries are retrieved from memory, but are user-modifiable here. Similarly, selection of the Continue button 736 displayed in panel 732 results in the display of an Invoiceables panel 740, which displays a similar list 742 of Invoiceables and invoiceable names and codes, Offered unit costs, Cap Amounts, and units, corresponding to the data provided during the budget template process described above, but these amounts are user-modifiable here, as will be appreciated from FIGS. 4AK and 4AL. This allows for customization of negotiations on a per-supplier basis, which is often necessary as a practical matter.

Selection of the Continue button 746 in the Invoiceables panel 740 results in display of the Negotiation Overview panel 752 as shown in FIG. 4AM. This panel, like the Template Overview panel 690 shown in FIGS. 4AG-4AH, shows Visits and Procedures and Invoiceables in tabular form, by Treatment Arm for the associated clinical trial study, with procedures/invoiceables displayed in rows, visits displayed in columns, and corresponding payment amounts being negotiated in cells of the table, as shown in FIG. 4AM. This provides a particularly compact and effective display of relevant information during negotiations.

The exemplary panel 752 displays a user selectable option 760 selectable to export/download the displayed budget information. The budget information may be downloaded in a spreadsheet or other file (e.g., PDF file) format. A PDF formatted report may be exported to allow for a clinical trial site/supplier to review the budget manually, outside of the CTFS system, as is commonly the practice now. In such a scenario, the supplier typically handwrites changes on the report and sends it back to the sponsor (e.g., using stored Sponsor Contact Data 224f), who may then enter changes into the CTFS system using graphical user interface windows displayed by the SAM 270.

In an alternative embodiment, the CTFS communicates with supplier Computing Devices 90b, 90c, and supplier users can interface directly with the CTFS system 200 via graphical user interface windows displayed by the SAM 270, so that the supplier user can review the clinical trial plan/budget and negotiate payment amounts electronically, within the CTFS system 200, without the need for export of data and manual entry of changes from a marked-up report. In this case, workflow automation may be used, e.g., to cause the CTFS 200 to send an email with a link allow the supplier user to login to the CTFS 200 to review the budget via graphical user interface windows displayed by the SAM at the Supplier Computing Device 90b, 90c.

Referring again to FIG. 3, if the payment offers for all tasks are approved by a particular supplier, as shown at 320, then a clinical trial agreement is effectively defined between the sponsor and the supplier, and the final study plan data/contract may be stored as Final Study Plan Data 224j, as shown at 330 in FIG. 3, and the clinical trial work may begin.

If, however, the payment offers for all tasks are not approved by a particular supplier, as shown at 320, then the CTFS 200 in this exemplary embodiment causes display of graphical user interface windows at the Supplier Computing Device 90b, 90c, to prompt for and receiving Supplier input in the nature of counteroffers, etc., as shown at 322. If agreement is not reach, further graphical user interface windows may be displayed (e.g., to the Sponsor) for negotiating pricing, as shown at 322, or the negotiation may simply be ended, as shown at 326 and 328. If agreement as to payments for all tasks is reached at 324, then a clinical trial agreement is effectively defined between the sponsor and the supplier, and the final study plan data/contract may be stored as Final Study Plan Data 224j, as shown at 330 in FIG. 3, and the clinical trial work may begin.

Accordingly, the negotiations between the sponsor and supplier may continue via electronic communications and/or interactions with the CTFS 200 until agreement is reached as to the clinical trial plan and/or payment amounts, at which point the sponsor may approve the negotiated plan and budget for the supplier. At this point, a clinical trial agreement is thereby effectively defined between the sponsor and the supplier, and the final study plan data/contract may be stored as Final Study Plan Data 224j, and the clinical trial work may begin.

Notably, the CTFS 200 may share/communicate this data to the CTPMS 305 electronically, and/or communicate with the PPS 405 to cause/initiate payments to the supplier to be made for at least one task, consistent with the approved clinical trial plan/pricing negotiated via the CTFS 200, via the PPS 405, as shown at 332. Additionally, the task and payment/budget information may be added to a data store of historical clinical trial payment data 224k/224a and be used to generate new FMV data for use to forecast and aid in the planning of future clinical trials. This avoids human/data entry errors and allows for particularly efficient and effective development of clinical trial study plans and negotiations relating to same, and effective building historical data-based fair market value data for use for forecasting purposes, as well as integration with payment systems to ensure accurate payments, and accurate capture of payment data for use on a going-forward basis.

The present invention may be operational with numerous other general-purpose or special-purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, cellular telephones, network PCs, minicomputers, mainframe computers, distributed computing environments that include any of the above-mentioned systems or devices, and the like.

The present invention has been described in the general context of computer-executable instructions, such as program modules or engines, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules/engines may be located in local and/or remote computer-storage media including, by way of example only, memory storage devices.

The exemplary computing system may include general-purpose computing hardware in the form of a server. Components of the server may include, without limitation, a processing unit, internal system memory, and a suitable system bus for coupling various system components, including a database cluster, with the server. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus.

The server typically includes therein, or has access to, a variety of computer-readable media, for instance, via a database cluster. Computer-readable media can be any available media that may be accessed by the server, and includes volatile and nonvolatile media, as well as removable and non-removable media. By way of example, and not limitation, computer-readable media may include computer-storage media and communication media. Computer-storage media may include, without limitation, volatile and nonvolatile media, as well as removable and non-removable media implemented in any method or technology for storage of information, such as computer readable instructions, data structures, program modules, or other data. In this regard, computer-storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server. Communication media typically embodies computer readable instructions, data structures, program modules, or other data in a modulated data signal, such as a carrier wave or other transport mechanism, and may include any information delivery media. The term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer-readable media.

The server may operate in a computer network using logical connections to one or more remote computers. Remote computers may be located at a variety of locations or over the Internet. The remote computers may be personal computers, servers, routers, network PCs, peer devices, other common network nodes, or the like, and may include some or all of the elements described above in relation to the server. The computing devices can be personal digital assistants or other like devices.

Exemplary computer networks may include, without limitation, local area networks (LANs) and/or wide area networks (WANs). Such networking environments are commonplace in offices, enterprise-wide computer networks, intranets, and the Internet. When utilized in a WAN networking environment, the server may include a modem/network card or other means for establishing communications over the WAN, such as the Internet. In a networked environment, program modules or portions thereof may be stored in the server, in the database cluster, or on any of the remote computers. For example, and not by way of limitation, various application programs may reside on the memory associated with any one or more of the remote computers. It will be appreciated by those of ordinary skill in the art that the network connections shown are exemplary and other means of establishing a communications link between the computers (e.g., the server and remote computers) may be utilized.

In operation, a user may enter commands and information into the server or convey the commands and information to the server via one or more of the remote computers through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote device to the server. In addition to a monitor, the server and/or remote computers may include other peripheral output devices, such as speakers and a printer.

Many other internal components of the server and the remote computers/computing devices are not shown because such components and their interconnection are well known. Accordingly, additional details concerning the internal construction of the server and the remote computers/computing devices are not further disclosed herein.

Although methods and systems of embodiments of the present invention may be implemented in a WINDOWS or LINUX operating system, operating in conjunction with an Internet-based delivery system, one of ordinary skill in the art will recognize that the described methods and systems can be implemented in any system supporting the functionality described herein. As contemplated by the language above, the methods and systems of embodiments of the present invention may also be implemented on a stand-alone desktop, personal computer, cellular phone, smart phone, tablet, PDA, or any other computing device used in various locations.

Additionally, computer readable media storing computer readable code for carrying out the method steps identified above is provided. The computer readable media stores code for carrying out subprocesses for carrying out the methods described herein.

A computer program product recorded on a computer readable medium for carrying out the method steps identified herein is provided. The computer program product comprises computer readable means for carrying out the methods described above.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A clinical trial forecasting system comprising:
a display device;
a user input device;
a memory operatively comprising a non-transitory data processor-readable medium;
a data processor operatively connected to the memory, the display device and the user input device;
user interface management instructions embodied in data processor-executable code stored in the memory, said user interface management instructions being executable by the data processor to provide a user interface display engine configured to:
receive and store, in the memory, historical payment data comprising data identifying payment amounts for a plurality of tasks for a plurality of clinical trials;
process historical payment data stored in the memory to perform at least one aggregation of the historical payment data for at least one of said plurality of tasks, said at least one aggregation representing a fair market value for said at least one of said plurality of tasks;
display, via the display device, at least one graphical user interface window operable to define a clinical trial study plan comprising a defined plurality of clinical trial tasks to be performed as part of a clinical trial; and
display, via the display device, at least one graphical user interface window operable to view fair market value data associated with at least one of the defined plurality of clinical trial tasks.

2. The clinical trial forecasting system of claim 1, further comprising user interface management instructions embodied in data processor-executable code stored in the memory and executable by the data processor to provide a user interface display engine configured to:
display, via the display device, at least one graphical user interface window operable to assign a respective proposed payment for at least one of the defined plurality of clinical trial tasks, to define a budget for the clinical trial;
receive at least one of a proposed change and an approval from a supplier's computing system for each proposed payment of the budget for the clinical trial; and
after receiving an approval for each of the defined plurality of clinical trial tasks, store data representing a clinical trial agreement identifying each of the defined plurality of clinical trial tasks and associated approved payments.

3. The clinical trial forecasting system of claim 1, further comprising user interface management instructions embodied in data processor-executable code stored in the memory and executable by the data processor to provide a user interface display engine configured to:
receive data confirming a supplier's performance of at least one of said defined plurality of clinical trial tasks;
retrieve stored data representing an associated clinical trial agreement's identification of an associated approved payment for said at least one of said defined plurality of clinical trial tasks; and
initiate payment to said supplier for performance of said at least one of said defined plurality of clinical trial tasks in an amount of the associated approved payment.

4. The clinical trial forecasting system of claim 3, wherein said instructions to initiate payment to said supplier comprises instructions to transmit data via a communications network to a payment processing system to cause payment to said supplier for performance of said at least one of said defined plurality of clinical trial tasks in said amount of said associated approved payment.
update the proposed payment for review by the supplier until approval for each proposed payment has been obtained; and

5. The clinical trial forecasting system of claim 1, wherein said instructions to display, via the display device, at least one graphical user interface window operable to define a clinical trial study plan comprises instructions to:
display, via the display device, at least one graphical user interface window operable to define a plurality of patient visits collectively defining a visit schedule for a clinical trial;
display, via the display device, at least one graphical user interface window operable to identify at least one procedure to define a list of selected procedures to be performed as part of the clinical trial;
display, via the display device, at least one graphical user interface window operable assign each procedure from the list of selected procedures to at least one visit of the visit schedule;
display, via the display device, at least one graphical user interface window operable to select at least one invoiceable task from a stored list of invoiceable tasks, to define a list of selected invoiceable tasks to be performed as part of the clinical trial;
display, via the display device, at least one graphical user interface window operable to assign each invoiceable task from the list of selected invoiceable tasks to at least one visit of the visit schedule;

6. The clinical trial forecasting system of claim 5, wherein said instructions to display, via the display device, at least one graphical user interface window operable to identify at least one procedure comprises instructions to:
retrieve a list of procedures from a set of stored procedures;
display at least a portion of the list of procedures via the display device; and
receive user input selecting at least one procedure from the displayed list of procedures.

7. The clinical trial forecasting system of claim 6, wherein said set of stored procedures comprises associated codes, and wherein said instructions to retrieve the list of procedures from the set of stored procedures comprises instructions to retrieve the list of procedures using at least one code received as user input.

8. The clinical trial forecasting system of claim 7, wherein the codes are CPT codes.

9. The clinical trial forecasting system of claim 7, wherein the codes are user-created codes.

10. The clinical trial forecasting system of claim 1, wherein said instructions to display, via the display device, at least one graphical user interface window operable to define a clinical trial study plan comprises instructions to:
retrieve, from the memory, at least one clinical trial study template; and
display, via the display device, at least one graphical user interface window displaying clinical trial study plan data retrieved from said at least one clinical trial study template, said clinical trial study plan data comprising the defined plurality of clinical trial tasks to be performed as part of a clinical trial; and
display, via the display device, at least one graphical user interface window permitting a user to modify at least one of the defined plurality of clinical trial tasks to be performed as part of a clinical trial and associated payments for the at least one of the defined plurality of clinical trial tasks.

11. The clinical trial forecasting system of claim 1, wherein said instructions to display, via the display device, at least one graphical user interface window operable to define a clinical trial study plan comprises instructions to:
display, via the display device, a plurality of at least one of patient visits, patient procedures and invoiceable tasks in tabular form, the at least one graphical user interface window displaying a user-manipulable element permitting reordering of items displayed in tabular form according to user input to the graphical user interface window.

12. The clinical trial forecasting system of claim 1, wherein said instructions to display, via the display device, at least one graphical user interface window operable to define a clinical trial study plan comprises instructions to:
display, via the display device, a warning icon in associated with any task that has not yet been mapped to EDC-based reporting from a clinical site.

13. The clinical trial forecasting system of claim 1, wherein said instructions to display, via the display device, at least one graphical user interface window operable to define a clinical trial study plan comprises instructions to:
display, via the display device, a user-manipulable element operable to turn on or off Overhead functionality that automated increases proposed payments by a specified percentage to account for facility overhead in pricing negotiations.

14. The clinical trial forecasting system of claim 1, wherein said instructions to display, via the display device, at least one graphical user interface window operable to assign a respective proposed payment comprises instructions to:
display, via the display device, at least one data entry field for receiving user input of at least one of a payment offer amount and a payment cap amount that the sponsor does not wish to exceed for payment for an associated task.

15. The clinical trial forecasting system of claim 1, wherein said instructions to display, via the display device, at least one graphical user interface window operable to view fair market value data associated with at least one of the defined plurality of clinical trial tasks comprises instructions to:
display, via the display device, a user-manipulable element operable to filter fair market value data, and to display the filtered data.

16. The clinical trial forecasting system of claim 1, wherein said instructions to display, via the display device, at least one graphical user interface window operable to view fair market value data associated with at least one of the defined plurality of clinical trial tasks comprises instructions to:
display, via the display device, a user-manipulable element operable to filter fair market value data by at least one of therapeutic area, indication, clinical trial study phase, country, currency, data source, and percentile, and to display the filtered data.

17. The clinical trial forecasting system of claim 1, wherein said instructions to display, via the display device, at least one graphical user interface window operable to view fair market value data associated with at least one of the defined plurality of clinical trial tasks comprises instructions to:
display, via the display device, a gauge in the nature of a qualitative display indicating whether an associated cost is low, medium, or high, based on a selected percentile for filtering of fair market value data.

18. The clinical trial forecasting system of claim 1, wherein said instructions to display, via the display device, at least one graphical user interface window operable to view fair market value data associated with at least one of the defined plurality of clinical trial tasks comprises instructions to:
display, via the display device, a single user-manipulable element operable to filter fair market value data for a plurality of clinical trial tasks according to input provided by the single user-manipulable element.

19. The clinical trial forecasting system of claim 1, further comprising user interface management instructions embodied in data processor-executable code stored in the memory and executable by the data processor to provide a user interface display engine configured to:
cause display, via a supplier's computing device separate from the clinical trial forecasting system, of at least one graphical user interface window displaying each respective proposed payment for at least one of the defined plurality of clinical trial tasks; and
cause display, via the supplier's computing device, a single user-manipulable element operable to receive user input indicating at least one of a proposed change and an approval for each respective proposed payment.

20. A method of controlling a display of a computerized device comprising a display device, a user input component, a memory operatively comprising a non-transitory data processor-readable medium, a data processor operative connected to the memory, the display and the user input component, and user interface management instructions embodied in data processor-executable code stored in the memory and executable by the data processor, the method comprising:
displaying, via the display device, at least one graphical user interface window operable to define a clinical trial study plan comprising a defined plurality of clinical trial tasks to be performed as part of a clinical trial;
displaying, via the display device, at least one graphical user interface window operable to assign a respective proposed payment for at least one of the defined plurality of clinical trial tasks, to define a budget for the clinical trial;
receiving at least one of a proposed change and an approval from a supplier's computing system for each proposed payment of the budget for the clinical trial; and
after receiving an approval for each of the defined plurality of clinical trial tasks, store data representing a clinical trial agreement identifying each of the defined plurality of clinical trial tasks and associated approved payments.

21. The method of claim 20, further comprising:
receiving and storing, in the memory, historical payment data comprising data identifying payment amounts for a plurality of tasks for a plurality of clinical trials;
processing historical payment data stored in the memory to perform at least one aggregation of the historical payment data for at least one of said plurality of tasks, said at least one aggregation representing a fair market value for said at least one of said plurality of tasks; and
displaying, via the display device, at least one graphical user interface window operable to view fair market value data associated with at least one of the defined plurality of clinical trial tasks.

22. The method of claim 20, further comprising:
receiving data confirming a supplier's performance of at least one of said defined plurality of clinical trial tasks;
retrieving stored data representing an associated clinical trial agreement's identification of an associated approved payment for said at least one of said defined plurality of clinical trial tasks; and
initiating payment to said supplier for performance of said at least one of said defined plurality of clinical trial tasks in an amount of the associated approved payment.

23. The method of claim 20, further comprising:
transmitting data via a communications network to a payment processing system to cause payment to a supplier for performance of said at least one of said defined plurality of clinical trial tasks in said amount of said associated approved payment.

24. The method of claim 20, further comprising:
retrieving, from the memory, at least one clinical trial study template; and
displaying, via the display device, at least one graphical user interface window displaying clinical trial study plan data retrieved from said at least one clinical trial study template, said clinical trial study plan data comprising the defined plurality of clinical trial tasks to be performed as part of a clinical trial; and
displaying, via the display device, at least one graphical user interface window permitting a user to modify at least one of the defined plurality of clinical trial tasks to be performed as part of a clinical trial and associated payments for the at least one of the defined plurality of clinical trial tasks.

25. The method of claim 20, further comprising:
displaying, via the display device, a plurality of at least one of patient visits, patient procedures and invoiceable tasks in tabular form, the at least one graphical user interface window displaying a user-manipulable element permitting reordering of items displayed in tabular form according to user input to the graphical user interface window.

26. The method of claim 20, further comprising:
displaying, via the display device, a warning icon in associated with any task that has not yet been mapped to EDC-based reporting from a clinical site.

27. The method of claim 20, further comprising:
displaying, via the display device, a user-manipulable element operable to turn on or off Overhead functionality that automated increases proposed payments by a specified percentage to account for facility overhead in pricing negotiations.

28. A computer program product for implementing a method of controlling a display of a computerized device, the computer program product comprising a non-transitory computer-readable medium storing executable instructions that, when executed by a processor, cause a computerized variable split allocation system to perform a method comprising:
displaying, via the display device, at least one graphical user interface window operable to define a clinical trial study plan comprising a defined plurality of clinical trial tasks to be performed as part of a clinical trial;
displaying, via the display device, at least one graphical user interface window operable to assign a respective proposed payment for at least one of the defined plurality of clinical trial tasks, to define a budget for the clinical trial;
receiving at least one of a proposed change and an approval from a supplier's computing system for each proposed payment of the budget for the clinical trial; and
after receiving an approval for each of the defined plurality of clinical trial tasks, store data representing a clinical trial agreement identifying each of the defined plurality of clinical trial tasks and associated approved payments.

29. The method of claim 28, further comprising:
receiving and storing, in the memory, historical payment data comprising data identifying payment amounts for a plurality of tasks for a plurality of clinical trials;
processing historical payment data stored in the memory to perform at least one aggregation of the historical payment data for at least one of said plurality of tasks, said at least one aggregation representing a fair market value for said at least one of said plurality of tasks; and
displaying, via the display device, at least one graphical user interface window operable to view fair market value data associated with at least one of the defined plurality of clinical trial tasks.

30. The method of claim 28, further comprising:
receiving data confirming a supplier's performance of at least one of said defined plurality of clinical trial tasks;
retrieving stored data representing an associated clinical trial agreement's identification of an associated approved payment for said at least one of said defined plurality of clinical trial tasks; and
initiating payment to said supplier for performance of said at least one of said defined plurality of clinical trial tasks in an amount of the associated approved payment.

31. The method of claim 28, further comprising:
transmitting data via a communications network to a payment processing system to cause payment to a supplier for performance of said at least one of said defined plurality of clinical trial tasks in said amount of said associated approved payment.

32. The method of claim 28, further comprising:
retrieving, from the memory, at least one clinical trial study template; and
displaying, via the display device, at least one graphical user interface window displaying clinical trial study plan data retrieved from said at least one clinical trial study template, said clinical trial study plan data comprising the defined plurality of clinical trial tasks to be performed as part of a clinical trial; and
displaying, via the display device, at least one graphical user interface window permitting a user to modify at least one of the defined plurality of clinical trial tasks to be performed as part of a clinical trial and associated payments for the at least one of the defined plurality of clinical trial tasks.

33. The method of claim 28, further comprising:
displaying, via the display device, a plurality of at least one of patient visits, patient procedures and invoiceable tasks in tabular form, the at least one graphical user interface window displaying a user-manipulable element permitting reordering of items displayed in tabular form according to user input to the graphical user interface window.

34. The method of claim 28, further comprising:
displaying, via the display device, a warning icon in associated with any task that has not yet been mapped to EDC-based reporting from a clinical site.

35. The method of claim 28, further comprising:
displaying, via the display device, a user-manipulable element operable to turn on or off Overhead functionality that automated increases proposed payments by a specified percentage to account for facility overhead in pricing negotiations.
